(19) United States
(12) Patent Application Publication
(10) Pub. No.: US 2026/0355154 A1
(43) Pub. Date: Nov. 12, 2026

(54) IMPLANTS, COMPOSITIONS, AND METHODS FOR TREATING RETINAL DISEASES AND DISORDERS

(71) Applicant: Osanni Bio, Inc., San Francisco, CA (US)

(72) Inventors: Chetan Pujara, Irvine, CA (US); John Paderi, San Francisco, CA (US); Yung-Wen Chiu, Mountain View, CA (US); Pinal Mistry, Richmond, TX (US); Eleonora Lad, Durham, NC (US); Douglas Michael Ackermann, Ross, CA (US)

(21) Appl. No.: 19/721,722

(22) Filed: Jun. 26, 2026

Related U.S. Application Data
(63) Continuation of application No. PCT/US2025/010208, filed on Jan. 3, 2025.
(60) Provisional application No. 63/618,224, filed on Jan. 5, 2024.

(51) Int. Cl. A61K 31/40; A61F 9/00; A61K 9/00; A61K 9/08; A61K 9/16; A61K 31/22; A61K 31/366; A61K 31/405; A61K 31/47; A61K 31/505; A61K 47/14

(52) U.S. Cl. CPC A61K 31/40; A61F 9/0017; A61K 9/0051; A61K 9/08; A61K 9/1647; A61K 31/22; A61K 31/366; A61K 31/405; A61K 31/47; A61K 31/505; A61K 47/14

(57) ABSTRACT

Provided are implants and methods for treating or preventing age-related macular regeneration (AMD) in patients in need thereof.

### CROSS-REFERENCE TO RELATED APPLICATIONS

This application claims the benefit under 35 U.S.C. § 119 (e) of U.S. Provisional Application Ser. No. 63/618,224, filed Jan. 5, 2024, the contents of which is hereby incorporated by reference in its entirety

### BACKGROUND

Age-related macular degeneration (AMD) is the leading cause of irreversible vision loss in adults in the Western world. AMD is broadly classified into two types, atrophic and neovascular.

The early stage of AMD (which is atrophic AMD) is characterized by the presence of a few medium-size drusen and pigmentary abnormalities such as hyperpigmentation or hypopigmentation of the retinal pigment epithelium (RPE). The intermediate stage of AMD is characterized by the presence of at least one of one large druse, numerous medium-size drusen, hyperpigmentation, and/or hypopigmentation of the RPE, either without signs of geographic atrophy (GA), or with GA that does not extend to the center of the macula (non-central [or para-central] GA). GA represents the absence of a continuous pigmented layer and the death of at least some portion of RPE cells. Non-central GA spares the fovea and thus preserves central vision.

The advanced stage of AMD is characterized by the presence of drusen and GA that extends to the center of the macula (central GA). Central GA includes macular atrophy. Central GA involves the fovea and thus results in significant loss of central vision and visual acuity.

The advanced stage of AMD that becomes “wet” AMD is characterized by neovascularization and any of its potential sequelae, including leakage (e.g., of plasma), plasma lipid and lipoprotein deposition, sub-RPE-BL, subretinal and intraretinal fluid, hemorrhage, fibrin, fibrovascular scars and RPE detachment. In choroidal neovascularization (CNV), new blood vessels grow up from the choriocapillaris and through the Bruch's membrane (BrM), which causes vision loss via the aforementioned sequelae. There are three types of neovascularization (NV). Type 1 NV occurs in the sub-RPE-BL space, and new blood vessels emanate from the choroid under the macular region. Type 2 NV occurs in the subretinal space above the RPE, and new blood vessels emanate from the choroid and break through to the subretinal space. In types 1 and 2 NV, new blood vessels cross the BrM and may ramify in the pro-angiogenic cleavage plane created by soft drusen and basal linear deposits (BLinD). Type 3 NV (retinal angiomatous proliferation) occurs predominantly within the retina (intraretinal), but can also occur in the subretinal space, and new blood vessels emanate from the retina with possible anastomoses to the choroidal circulation. Type 3 NV is the most difficult subtype of NV to diagnose and has the most devastating consequences in terms of photoreceptor damage, but type 3 NV responds well to treatment with an anti-VEGF agent. A neovascular AMD patient can also have a mixture of subtypes of NV, including type 1 plus type 2, type 1 plus type 3, and type 2 plus type 3. The approximate occurrence of the different subtypes of NV among newly presenting neovascular AMD patients is: 40% type 1, 9% type 2, 34% type 3, and 17% mixed (of the mixed, 80% type 1 plus type 2, 16% type 1 plus type 3, and 4% type 2 plus type 3). Another form of NV is polypoidal vasculopathy, which is of choroidal origin and is the most common form of NV among Asian populations, whose eyes generally have few drusen but may have BLinD. The RPE can become detached from the BrM in each subtype of NV. For instance, leakage of fluid from neovessels into the sub-RPE-BL space in type 1 NV can result in pigment epithelium detachment. The new blood vessels generated by NV are fragile, leading to leakage of fluid, blood and proteins below the macula. Leakage of blood into the subretinal space is particularly toxic to photoreceptors, and intraretinal fluid signifies a poor prognosis for vision. Bleeding and leaking from the new blood vessels, with subsequent fibrosis, can cause irreversible damage to the retina and rapid vision loss if left untreated.

### SUMMARY

The present disclosure, in one embodiment, provides extended release unit dosage forms for ocular administration of a statin. In some embodiments, the extended release unit dosage form is an ocular implant. In some embodiments, the extended release unit dosage form is a flowable composition.

Accordingly, in some embodiments, provided herein is an ocular implant comprising a statin and a polymer matrix, wherein the statin is dispersed in the polymer matrix and the polymer matrix controls release of the statin.

In some embodiments, provided herein is a flowable composition comprising a statin and a solvent or excipient, wherein the statin is dispersed in a solvent or excipient and the solvent or excipient controls release of the statin.

In some embodiments, provided herein is a method of treating or preventing age-related macular degeneration (AMD), comprising administering a statin to a patient in need thereof, wherein the statin is administered to the eye in an extended release unit dosage form. In some embodiments, the statin concentration within the eye is substantially maintained at a concentration of greater than about 1 nM, or about 2 nM, or about 3 nM, or about 4 nM, or about 5 nM, or about 6 nM, or about 7 nM, or about 8 nM, or about 9 nM, or about 10 nM, for the duration of a treatment period, such as at least 1 month, or 3 months, or 6 months, or 1 year. In some embodiments, the statin concentration within the eye refers to the intravitreal concentration of statin within one of the eyes. In some embodiments, provided herein is a method of treating or preventing age-related macular degeneration (AMD), comprising administering a statin to a patient in need thereof, wherein the statin is administered to the eye in an extended release unit dosage form. In some embodiments, the statin concentration within the eye is substantially maintained at a concentration of greater than about 10 nM for the duration of a treatment period, such as at least 1 month, or 3 months, or 6 months, or 1 year. In some embodiments, the statin concentration within the eye refers to the intravitreal concentration of statin within one of the eyes.

In some embodiments, both eyes are treated. In some embodiments, one eye of a patient is treated.

In some embodiments, provided herein is a method of treating or preventing age-related macular degeneration (AMD) comprising administering a statin to a patient in need thereof, wherein the administering is by intravitreal injection.

In some embodiments, provided herein is a method of reducing drusen size and/or number comprising administering a statin to a patient in need thereof, wherein the administering is by intravitreal injection.

In some embodiments, provided herein is a method of preventing, reducing, or reversing complement activation in the eye comprising administering a statin to a patient in need thereof, wherein the administering is by intravitreal injection.

The implants and compositions provided herein can be used employed to treat or prevent one or more of the following indications (e.g., retinal diseases and disorders), such as, but not limited to, age-related macular degeneration, macular drusen (small, intermediate, large), peripheral drusen, extramacular drusen, drusenoid pigment epithelial detachment (PED), drusenoid deposits, basal laminar deposits, basal linear deposits, doyne honeycomb retinal dystrophy, Malattia Leventinese, familial dominant drusen (or autosomal dominant drusen), cuticular drusen, serous detachment of RPE, drupelets, RPE atrophy, geographic atrophy, ellipsoid zone (EZ) attenuation, EZ loss, incomplete retinal pigment epithelial and outer retinal atrophy (iRORA), complete retinal pigment epithelial and outer retinal atrophy (cRORA), nascent geographic atrophy, retinal flecks, fundus flavimaculatus, Best disease, adult-onset vitelliform macular dystrophy, Best vitelliform macular dystrophy, autosomal recessive bestrophinopathy, vitelliform material, pattern dystrophy, autosomal dominant vitreoretinochoroidopathy, BEST1 gene mutation disorders, retinal emboli (in retinal artery occlusion), retinal exudates, retinal exudates secondary to retinal microaneurysm, familial exudative vitreoretinopathy (FEVR), synchysis scintillans (cholesterolosis bulbi), neuronal ceroid lipofuscinosis, Batten's Disease, retinitis pigmentosa, Bietti's crystalline dystrophy, juvenile macular degeneration (e.g., Stargardt disease), macular telangiectasia, maculopathy (e.g., age-related maculopathy (ARM) and diabetic maculopathy (DMP) (including partial ischemic DMP)), macular edema (e.g., diabetic macular edema (DME) (including clinically significant DME, focal DME and diffuse DME), Irvine-Gass Syndrome (postoperative macular edema), and macular edema following RVO (including central RVO and branch RVO)), retinopathy (e.g., diabetic retinopathy (including in patients with DME), Purtscher's retinopathy and radiation retinopathy), retinal artery occlusion (RAO) (e.g., central and branch RAO), retinal vein occlusion (RVO) (e.g., central RVO (including central RVO with cystoid macular edema (CME)) and branch RVO (including branch RVO with CME)), glaucoma (including low-tension, normal-tension and high-tension glaucoma), ocular hypertension, retinitis (e.g., Coats' disease (exudative retinitis) or retinitis pigmentosa), chorioretinitis, choroiditis (e.g., serpiginous choroiditis), uveitis (including anterior uveitis, intermediate uveitis, posterior uveitis with or without CME, and pan-uveitis), retinal detachment (e.g., in von Hippel-Lindau disease), retinal pigment epithelium (RPE) detachment, bestrophinopathy, Doyne honeycomb/dominant drusen, and diseases associated with increased intra- or extracellular lipid storage or accumulation in addition to AMD.

The implants and compositions of the present disclosure may also be used for treating an ocular disease is selected from the group consisting of glaucoma, diabetic retinopathy (DR), retinal vein occlusion (RVO), and retinopathy of prematurity (ROP).

### BRIEF DESCRIPTION OF DRAWINGS

FIG. 1 shows in vitro release profiles for ocular implants using atorvastatin calcium salt (Formulation 1 and Formulation 2) and atorvastatin free acid (Formulation 3 and Formulation 4) using hot melt extrusion processes.

FIG. 2 shows in vitro release profiles for atorvastatin in benzyl benzoate.

FIG. 3 shows results of a drusen assay using atorvastatin formulations.

FIG. 4 shows results complement activity using atorvastatin formulations.

FIG. 5 shows in vitro release of implant formulations listed in Table 3A.

FIG. 6 shows in vitro release of implant formulations listed in Table 3B and Table 3C.

FIG. 7 shows in vitro release of implant formulations listed in Table 4.

### DETAILED DESCRIPTION

All numerical designations, e.g., pH, temperature, time, concentration, and molecular weight, including ranges, are approximations which are varied (+) or (−) by increments of 0.1 or 20%, or 10%. It is to be understood, although not always explicitly stated that all numerical designations are preceded by the term “about”. Reference to “about” a value or parameter herein includes (and describes) embodiments that are directed to that value or parameter per se. In certain embodiments, the term “about” includes the indicated amount ±20%. In certain embodiments, the term “about” includes the indicated amount ±10%. In other embodiments, the term “about” includes the indicated amount ±5%. In certain other embodiments, the term “about” includes the indicated amount ±1%. It also is to be understood, although not always explicitly stated, that the reagents described herein are merely exemplary and that equivalents of such are known in the art.

As used herein, an “ocular implant” or “implant” refers to a solid device, which is structured, sized, or otherwise configured, to be delivered to an eye. Ocular implants in accordance with the present disclosure are generally biocompatible with physiological conditions of an eye and may not cause adverse side effects or immunological reaction. Ocular implants may be placed in an eye without disrupting vision of the eye. Non-limiting examples include extruded filaments or rods having a diameter and cut to a length suitable for placement in an ocular region of the eye, such as the posterior chamber. In some embodiments, the implants are biodegradable. In some embodiments, the ocular implant is suitable for intravitreal injection (or intravitreal implantation).

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of ocular regions in the eye include the anterior chamber, the posterior chamber, the vitreous cavity, the vitreous body, the choroid, the suprachoroidal space, the conjunctiva, the subconjunctival space, the sub-tenon space, the episcleral space, the intracorneal space, the epicorneal space, the sclera, the pars plana, surgically-induced avascular regions, the macula, and the retina.

An “intravitreal” implant is an implant that is sized for placement in the vitreous body of the eye. The ocular implants disclosed herein are typically syringeable.

As used herein, a “polymer” is intended to encompass both homopolymers (polymers having only one type of repeating unit) and copolymers (a polymer having more than one type of repeating unit).

As used herein, a “polymer matrix” refers to a substantially homogeneous mixture of polymers. In other words, the matrix does not include a mixture wherein one portion thereof is different from the other portion by ingredient, density, and etc. The mixture of polymers may be of the same type, e.g. two different PLA polymers, or of different types, e.g. PLA polymers combined with PLGA polymers.

As used herein, a “flowable composition” refers to a pharmaceutical composition having a consistency that allows the composition to flow readily (e.g., a liquid). The flowable composition can be a solution (e.g., the statin is dissolved in a carrier) or a suspension (i.e., particles in a carrier).

As used herein, “dispersed” means that the statin as disclosed herein is distributed, mixed, suspended, dissolved, and/or homogenized, within the polymer matrix of the ocular implant or the solvent of the flowable composition (e.g., benzyl benzoate). As such, the term dispersed includes solutions, emulsions, suspensions, and other dispersed systems.

“Substantially” in relation to the release profile or the release characteristic of statin means that the rate of release (i.e. amount of statin released/unit of time) does not vary by more than 100%, or by more than 50%, over a treatment period. “Substantially” in relation to the blending, mixing or dispersing of an active agent (i.e., statin) in a polymer, as in the phrase “substantially homogenously dispersed” means that there is a small difference in concentration of statin throughout the polymer matrix or solvent of the composition (e.g., a homogenous dispersal).

The term “biodegradable,” as used herein, means that the ocular implant or flowable composition is capable of being broken down into innocuous products in the normal functioning of the body.

The term “non-biodegradable,” as used herein, means that the ocular implant or flowable composition is not capable of being broken down in the body.

A “pharmaceutical composition” is intended to include the combination of one or more active agents (e.g., statin) with one or more carriers, inert or active, making the composition suitable for therapeutic use in vitro, in vivo or ex vivo.

“An effective amount” refers to the amount of an agent sufficient to induce a desired biological and/or therapeutic result. That result can be alleviation of the signs, symptoms, or causes of a disease, or any other desired alteration of a biological system.

As used herein, the terms “treating,” “treatment,” and the like are used herein to mean obtaining a desired pharmacologic and/or physiologic effect. The effect may be prophylactic in terms of completely or partially preventing a disorder or sign or symptom thereof, and/or may be therapeutic in terms of a partial or complete cure for a disorder and/or adverse effect attributable to the disorder.

### Extended Release Unit Dosage Forms

Provided herein are extended release unit dosage forms for ocular administration of a statin, comprising an ocular implant or flowable composition as described herein. Extended release unit dosage forms release statin from the ocular implant or flowable composition at a sustained rate. can either be sustained release or controlled release unit dosage forms.

In some embodiments, the extended release unit dosage form is a sustained release unit dosage form, where the unit dosage form maintains statin release (or administration) over a sustained period, but not at a constant rate.

Sustained release implies that the active agent is not released from the implant sporadically, in an unpredictable fashion. The term “sustained release” may include a partial “burst phenomenon” associated with deployment. In some example embodiments, an initial burst of statin may be desirable, followed by a more gradual release thereafter. The release rate may be steady state (commonly referred to as “timed release” or zero order kinetics), that is the statin is released in even amounts over a predetermined time (with or without an initial burst phase), or may be a gradient release. Sustained release compositions have substantially constant release over a given time period.

In some embodiments, the extended release unit dosage form is a controlled release unit dosage form, where the unit dosage form maintains statin release (or administration) over a sustained period at a nearly constant rate.

Controlled release drug delivery systems are classified based on the mechanism of drug release from the dosage form into dissolution controlled, diffusion-controlled, water penetration-controlled (osmotic pressure-controlled and swelling-controlled), chemically controlled and nanoparticle-based systems.

Dissolution Controlled Drug Delivery Systems: In dissolution-controlled release systems, drugs are either coated with or encapsulated within slowly dissolving polymeric membranes (reservoir systems) or matrices (monolithic systems), respectively. In reservoir systems, drugs are protected inside polymeric membranes with low solubility, where the rate-limiting step is dissolution.

Diffusion-Controlled Drug Delivery Systems: In diffusion-controlled release systems, drugs are trapped in and released via diffusion through inert water-insoluble polymeric membranes (reservoir systems) or polymeric matrices (monolithic systems). These are classified into membrane control reservoir systems and monolithic matrix systems. The drug release is governed by Fick's laws of diffusion. The rate-limiting step in diffusion-controlled systems is the diffusion of drugs. Diffusion-controlled systems are classified into membrane-controlled and monolithic or matrix systems. In membrane-controlled systems, the drug is contained in the core as a reservoir and is covered by a thin polymeric membrane. The membrane could be either porous or non-porous. The release of drugs is by diffusion through the membrane and the rate of release is governed by membrane thickness, porosity and physicochemical characteristics of drugs (partition coefficient, molecular size and diffusivity, protein binding and dosage). A common method to fabricate membrane-controlled reservoir systems includes encapsulation. The drug release is through diffusion when the outside layer that is exposed to the solution gets dissolved first, allowing drugs to diffuse out of the matrix.

In monolithic or matrix-controlled delivery systems, the drug is dispersed in a polymer matrix or solvent (e.g., benzyl benzoate). In monolithic systems, where a drug is dissolved, drugs are loaded below the solubility limit. As the size of the matrix decreases, the drug released decreases. Here the drug release is typically nonzero order, i.e., rate of absorption rate of elimination. In monolithic systems where the drugs are dispersed in the polymer matrix or solvent, drugs are typically loaded above the solubility limit.

Water Penetration-Controlled Drug Delivery Systems: These are classified as osmotic pressure-controlled drug delivery systems and swelling controlled drug delivery systems. The rate control is dependent on water penetration into the system.

Osmotic Controlled Drug Delivery Systems: Osmotic drug delivery uses the osmotic pressure for controlled delivery of drugs by using osmogens. Osmosis refers to the process of movement of solvent from a lower concentration of solute towards a higher concentration of solute across the semipermeable membrane. Osmotic pressure is the pressure exerted by the flow of water through a semipermeable membrane separating two solutions with different concentrations of solute. These systems can be used for administration via injection.

Basic components of osmotic drug delivery systems include the drug which itself may act as osmogen; otherwise, osmogenic salt can be added to the formulation. A semipermeable membrane with sufficient wet strength and water permeability that is biocompatible and rigid in withstanding the pressure within the device is needed. Apart from that, an outer coating material that is permeable to water but impermeable to solute can be used. Polymers such as cellulose acetate, cellulose triacetate and ethyl celluloses are commonly used in osmotic drug delivery systems. The advantages of osmotic-controlled delivery systems include increased efficacy of the drug, controlled drug delivery and reduced dosing frequency. A simple osmotic delivery system is a pump that is made up of two compartments separated by a moving partition. Compartment one is filled with an osmotic agent covered by a semi-permeable membrane. Compartment 2 is covered by a hard rigid shell with a delivery orifice.

Swelling-Controlled Drug Delivery Systems: In swelling-controlled drug delivery systems, the drug is dispersed or dissolved in the hydrophilic polymer when in a glassy (hard and rigid) state. In an aqueous environment, such as a physiological environment, or biological fluid (e.g., vitreous humor) liquid penetrates and swells the polymer matrix, which results in slow drug diffusion out of the polymer matrix. In certain embodiments, the polymer matrix comprises a hydrogel.

Chemically Controlled Drug Delivery Systems: Chemically controlled delivery systems change their chemical structure when exposed to the biological milieu. These are typically made primarily from biodegradable polymers which degrade in the body as a result of natural biological processes, eliminating the need to remove the delivery system after exhausting an active agent from the system. These are classified into two types: Polymer-drug dispersion system and polymer-drug conjugate systems. In polymer-drug dispersion systems, the drug is dispersed (e.g., suspended or dissolved) in a biodegradable polymer matrix and released through degradation of polymers under physiological conditions. Two types of biodegradations are reported: bulk erosion, which is through breakdown of polymers in the bulk and, surface erosion which is due to the breakdown of polymers from the surface or dissolution of polymers from the surface. Various factors that affect degradation (bioerosion and bulk erosion) include chemical structure and composition, the presence of unexpected units or chain defects, configuration, and molecular weight.

The extended release unit dosage forms described herein minimize the frequency of intravitreal statin administration. In some embodiments, the extended release unit dosage form is configured to substantially maintain a desired intravitreal concentration of statin over the course of a treatment period. In some embodiments, the intravitreal concentration of statin is substantially maintained at least about 10 nM, or from about 10 nM to about 5 μM, or about 100 nM, over the course of the treatment period. In some embodiments, the treatment period is 1-30 days, 4-52 weeks, 1-12 months, or 1-5 years.

In some embodiments, the intravitreal concentration of statin is substantially maintained at least about 1-10 μg/mL over the course of the treatment period. In some embodiments, the treatment period is 1-30 days, 4-52 weeks, 1-12 months, or 1-5 years.

In some embodiments, to achieve the required therapeutic concentration of the statin and to maintain the concentration of the statin for a desired treatment period, the unit dosage form is made up of two parts. The first which contains a loading dose and the second part which contains a maintenance dose. The desired response of the statin is achieved by the loading dose (the initial burst dose causes a rapid onset of the pharmacological effect) and the maintenance dose release of the statin is administered at a slow and steady rate (e.g., following the zero-order kinetics) to maintain the pharmacological effect of the statin. In some embodiments, the rate of maintenance dose at which a certain statin is administered substantially equals the rate of the drug output, however, this is not required in the extended release unit dosage forms described herein.

### Statins

The term “statin” as used herein is intended to refer to compounds that inhibit the enzyme HMG-CoA reductase by competitively binding to HMG-CoA reductase in the HMG-COA active site and/or disrupt the HMG-COA reductase pathway. Non-limiting examples of statins include atorvastatin (LIPITOR®), cerivastatin, fluvastatin (LESCOL®), lovastatin (MEVACOR®, ALTOCOR™), pitavastatin (UVALOR), pravastatin (PRAVACHOL®, SELEKTINE®), rosuvastatin (CRESTOR®) simvastatin (ZOCOR®), cerivastatin, or analogs thereof, or a combination thereof.

Statins can be either lipophilic or hydrophilic. Lipophilic statins include, for example, atorvastatin, lovastatin, and simvastatin. Hydrophilic statins include, for example, fluvastatin, rosuvastatin, and pravastatin. In some embodiments, a statin is lipophilic (e.g., atorvastatin). In some embodiments, the active agent is a HMG-COA reductase inhibitor. The HMG-COA reductase inhibitor may be any HMG-CoA reductase inhibitor capable of lowering plasma concentrations of low-density lipoprotein, total cholesterol, or both. Examples of HMG-COA reductase inhibitors that may be used include, but are not limited to, lovastatin (MEVACOR®); see U.S. Pat. Nos. 4,231,938; 4,294,926; 4,319,039), simvastatin (ZOCOR®; see U.S. Pat. Nos. 4,444,784; 4,450,171, 4,820,850; 4,916,239), pravastatin (PRAVACHOL®; see U.S. Pat. Nos. 4,346,227; 4,537,859; 4,410,629; 5,030,447 and 5,180,589), lactones of pravastatin (see U.S. Pat. No. 4,448,979), fluvastatin (LESCOL®; see U.S. Pat. Nos. 5,354,772; 4,911,165; 4,739,073; 4,929,437; 5,189,164; 5,118,853; 5,290,946; 5,356,896), lactones of fluvastatin, atorvastatin (LIPITOR®; see U.S. Pat. Nos. 5,273,995; 4,681,893; 5,489,691; 5,342,952), lactones of atorvastatin, cerivastatin (also known as rivastatin and BAYCHOL®; see U.S. Pat. No. 5,177,080, and European Application No. EP-491226A), lactones of cerivastatin, rosuvastatin (Crestor®; see U.S. Pat. Nos. 5,260,440 and RE37314, and European Patent No. EP521471), lactones of rosuvastatin, itavastatin, nisvastatin, visastatin, atavastatin, bervastatin, compactin, dihydrocompactin, dalvastatin, fluindostatin, pitivastatin, mevastatin (see U.S. Pat. No. 3,983,140), and velostatin (also referred to as synvinolin). Other examples of HMG-COA reductase inhibitors are described in U.S. Pat. Nos. 5,217,992; 5,196,440; 5,189,180; 5,166,364; 5,157,134; 5,110,940; 5,106,992; 5,099,035; 5,081,136; 5,049,696; 5,049,577; 5,025,017; 5,011,947; 5,010,105; 4,970,221; 4,940,800; 4,866,058; 4,686,237; 4,647,576; European Application Nos. 0142146A2 and 0221025A1; and PCT Application Nos. WO 86/03488 and WO 86/07054.

The term “statin” as used herein includes also pharmaceutically acceptable salts, esters, metabolites, hydrates, polymorphs, or crystals thereof, as well as lactones or the corresponding open dihydroxy acid.

Statins may exist in a non-salt form (e.g., a free base or a free acid, or having no basic or acidic atom or functional group) or as a salt. If a compound has, e.g., a basic nitrogen atom, the compound may form an addition salt with an acid (e.g., a mineral acid (such as HCl, HBr, HI, nitric acid, phosphoric acid or sulfuric acid) or an organic acid (such as a carboxylic acid or a sulfonic acid)). Suitable acids for use in the preparation of pharmaceutically acceptable salts include without limitation acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, D-gluconic acid, D-glucuronic acid, L-glutamic acid, alpha-oxo-glutaric acid, glycolic acid, hippuric acid, hydrobromic acid, hydrochloric acid, hydroiodic acid, (+)-DL-lactic acid, (+)-L-lactic acid, lactobionic acid, lauric acid, maleic acid, (−)-L-malic acid, malonic acid, (+)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, propionic acid, L-pyroglutamic acid, pyruvic acid, saccharic acid, salicylic acid, 4-amino-salicylic acid, sebacic acid, stearic acid, succinic acid, sulfuric acid, tannic acid, (+)-DL-tartaric acid, (+)-L-tartaric acid, thiocyanic acid, p-toluenesulfonic acid, undecylenic acid, and valeric acid.

If a compound has an acidic group (e.g., a carboxyl group), the compound may form an addition salt with a base. Pharmaceutically acceptable base addition salts can be formed with, e.g., metals (e.g., alkali metals or alkaline earth metals) or amines (e.g., organic amines). Non-limiting examples of metals useful as cations include alkali metals (e.g., lithium, sodium, potassium and cesium), alkaline earth metals (e.g., magnesium and calcium), aluminum and zinc. Metal cations can be provided by way of, e.g., inorganic bases, such as hydroxides, carbonates and hydrogen carbonates. Non-limiting examples of organic amines useful for forming base addition salts include chloroprocaine, choline, cyclohexylamine, dibenzylamine, N,N′-dibenzylethylenediamine, dicyclohexylamine, diethanolamine, ethylenediamine, N-ethylpiperidine, histidine, isopropylamine, N-methylglucamine, procaine, pyrazine, triethylamine and trimethylamine. Pharmaceutically acceptable salts are discussed in detail in Handbook of Pharmaceutical Salts, Properties, Selection and Use, P. Stahl and C. Wermuth, Eds., Wiley-VCH (2011).

In some embodiments, the statin is atorvastatin. Atorvastatin, as used herein, may be present in the form of atorvastatin or pharmaceutically acceptable salts thereof, for example, calcium, magnesium, or potassium. Atorvastatin may exist in any of the solid state forms available such as amorphous, or any other polymorphic form. As used herein, “atorvastatin” includes atorvastatin itself, as well as pharmaceutically acceptable salts of atorvastatin (alkali metal salts such as a sodium salt and a potassium salt; alkaline earth metal salts such as a calcium salt and a magnesium salt; organic amine salts such as a phenethylamine salt; ammonium salts, and the like), as well as solvates of atorvastatin or the pharmaceutically acceptable salts thereof with water, alcohol, or the like, and more than one of these can be used in combination. In some embodiments, the statin is present in the free dihydroxy acid form and optionally partly in the alkali metal, alkaline earth metal, ammonium and/or magnesium salt form and/or salt form with the further component as the ammonium cation.

Various physical forms (e.g., stereoisomers, stereoisomer mixtures, enantiomers, solvates, hydrates, isomorphs, pseudomorphs, polymorphs, salt forms, and combinations thereof) of statins (e.g., atorvastatin salts and polymorphs) are provided herein. For example, for use in the implants and compositions disclosed herein include physical forms of atorvastatin calcium salt, including polymorphs, solvates, and hydrates. Various physical forms of atorvastatin calcium salt are described in Jin, Herr M. Sc Yong Suk. “Discovering New Crystalline Forms of Atorvastatin Calcium-New Strategies for Screening.” (2012); in PCT Application Publication Nos. WO2002/043732, WO2001/036384, WO2006/106372, WO2004/050618, WO1997/003959, WO2003/004470, WO2009/007856, WO2003/050085, WO2007/096903, WO2006/011041, WO2005/090301, WO2005/092852, WO2012/015157, WO2008/002655, WO2006/012499, WO2002/057229, WO1997/003958, WO2008/089557, WO2007/070667, WO2003/011826, WO2002/051804, WO2004/022053, WO2006/048894, and WO2003/070702; in Rao, V. Pandu Ranga, et al. “Preparation of stable new polymorphic form of atorvastatin calcium.” Pharm. Lett 3.5 (2011): 48-53; in Ahn, S. G., Lee, H. W., Yoo, C. L., Kim, Y. M., Song, C. G., Kang, S. K., Kim, D. J., Soh, B. K., Nam, D. H., Shin, H. J., Novel crystal atorvastatin hemicalcium which is an inhibitor of HMG-COA reductase, KR 1020090090942, 2009; in An, Su-Gyeong, and Young-Taek Sohn. “Crystal forms of atorvastatin.” Archives of pharmacal research 32 (2009): 933-936; in Cho, D. O., Kim, S. K., Kim, B. R., Method for preparing a novel crystalline type of atorvastatin hemi-calcium, KR 1020110087026, 2011; and in Jin, Y. S., Ulrich, J., New crystalline solvates of atorvastatin calcium, in: Louhi-Kultanen, M., Hatakka, H., (Ed.), Proceedings of the 16th Symposium on Industrial Crystallization, Lappeenranta, Finland, (2009) 45-52, the contents of which are incorporated herein by reference in their entirety. The atorvastatin calcium salt can be or comprise a form selected from the group consisting of I, II, III, IV, V, VI, VII, VIII, IX, X, XI, XII, XIII, XIV, XV, XVI, XVII, XVIII, XIX, XX, XXI, XXII, XXIII, XXIV, XXV, XXVI, XXVII, XXVIII, XXIX, XXX, V(T), VI(T), VII(T), VIII(T), IX(T), X(T), XI(T), XIa(T), XII(T), XIV(T), XVI(T), XVII(T), XVIII(T), XIX(T), V(B), VI(R), VII(R), X(C), α, A, A1, B1, B2, B-52, C, D, E, F, Fa, Ga, Je, M, M-1, M-2, M-3, M-4, MCK-I, MCK-II, MCK-III, MD-1, MD-2, P, R, R2, T1, T2, T3, 3, or any combination thereof.

In some embodiments, the statin is atorvastatin free acid.

In some embodiments, the statin is atorvastatin calcium salt.

In some embodiments, the statin has low solubility in the flowable composition or polymer matrix of an ocular implant. Accordingly, the statin can be milled or pelleted in solid form and dispersed in the carrier (e.g., benzyl benzoate or polymer). In some embodiments, the average size of the active agent is 2 μm or less, 1.5 μm or less, or 1 μm or less, in size.

In some embodiments, the atorvastatin calcium salt is present in particles, which particles are dispersed in the polymer matrix or the benzyl benzoate.

In some embodiments, the statin has high solubility in either the solvent(s) or excipient(s) of the flowable composition or the polymer matrix. In instances where the statin is soluble in benzyl benzoate, sterilization may be performed via filtration, which then avoids terminal sterilization by means such as gamma irradiation or e-beam.

### Ocular Implants

In some embodiments, provided herein is an ocular implant comprising a statin and a polymer matrix, wherein the statin is dispersed (i.e., dissolved or suspended) in the polymer matrix. In the ocular implants, the polymer matrix is what controls release, and thus administration, of the statin.

It is contemplated that any polymer can be used to provide the polymer matrix, provided that the polymer matrix is biocompatible, specifically biocompatible within the eye, e.g., the vitreous humor. In some embodiments, suitable polymers for use in the polymer matrix includes polymers used in extended release drug delivery systems, such as synthetic polymers, natural polymers, or stimuli-responsive polymers.

Exemplary, non-limiting, synthetic polymers include polyhydroxy ethyl methacrylate poly(2-hydroxyethyl methacrylate), ethyl cellulose, hydroxypropyl methyl cellulose (HPMC), eudragits, polylactic acid (PLA), polylactic-co-glycolic acid (PLGA), polycaprolactone, polyvinyl Pyrrolidone (PVP), poly methyl methacrylate (PMMA), poly-(N-Isopropyl acrylamide) (PNIPAM), poly(ethylenimine), cyclodextrin (α, β, γ), or carbomers.

Exemplary, non-limiting, natural polymers include alginates, starches, dextrans, cellulose, gums (acacia, tragacanth, guar gum), chitosan, hyaluronic acid, collagen, gelatine, microbial polymers (polyhydroxy butyrate), or arginine derivatives.

Exemplary, non-limiting, stimuli-responsive polymers include pH-responsive polymers (e.g., polyacids (such as PLA, polymethacrylate, poly aspartate, alginates, polystyrene sulphonic acid, and the like), polybases (such as chitosan, poly-L-lysine, polyallylamine, polyethylene amine, polyamidoamine dendrimer, and the like), thermoresponsive polymers (e.g., poly-(N-isopropyl acrylamide) (PNIPAM), poly-(N-vinylcaprolactam), poly(N,N-dimethyl acrylamide), poly(methyl vinyl ether), and the like), electric responsive polymers (e.g., sulfonated polystyrenes, poly(thiophene) s, poly(ethyl oxazoline) s, and the like), ultrasound responsive polymers (e.g., ethylene-vinyl acetate), or light responsive polymers (e.g., modified poly(acrylamide) s).

In some embodiments, the ocular implant is non-biodegradable.

In some embodiments, the polymer matrix is swellable in vivo.

In some embodiments, the ocular implant is biodegradable.

Non-limiting examples of biodegradable polymers include polyesters, poly(Į-hydroxyacids), polylactide, polyglycolide, poly(İ-caprolactone), polydioxanone, poly(hydroxyalkanoates), poly(hydroxypropionates), poly(3-hydroxypropionate), poly(hydroxybutyrates), poly(3-hydroxybutyrate), poly(4-hydroxybutyrate), poly(hydroxypentanoates), poly(3-hydroxypentanoate), poly(hydroxyvalerates), poly(3-hydroxyvalerate), poly(4-hydroxyvalerate), poly(hydroxyoctanoates), poly(2-hydroxyoctanoate), poly(3-hydroxyoctanoate), polysalicylate/polysalicylic acid, polycarbonates, poly(trimethylene carbonate), poly(ethylene carbonate), poly(propylene carbonate), tyrosine-derived polycarbonates, L-tyrosine-derived polycarbonates, polyiminocarbonates, poly(DTH iminocarbonate), poly(bisphenol A iminocarbonate), poly(amino acids), poly(ethyl glutamate), poly(propylene fumarate), polyanhydrides, polyorthoesters, poly(DETOSU-1,6HD), poly(DETOSU-t-CDM), polyurethanes, polyphosphazenes, polyimides, polyamides, nylons, nylon 12, polyoxyethylated castor oil, poly(ethylene glycol), polyvinylpyrrolidone, poly(L-lactide-co-D-lactide), poly(L-lactide-co-D,L-lactide), poly(D-lactide-co-D,L-lactide), poly(lactide-co-glycolide), poly(lactide-co-İ-caprolactone), poly(glycolide-co-İ-caprolactone), poly(lactide-co-dioxanone), poly(glycolide-co-dioxanone), poly(lactide-co-trimethylene carbonate), poly(glycolide-co-trimethylene carbonate), poly(lactide-co-ethylene carbonate), poly(glycolide-co-ethylene carbonate), poly(lactide-co-propylene carbonate), poly(glycolide-co-propylene carbonate), poly(lactide-co-2-methyl-2-carboxyl-propylene carbonate), poly(glycolide-co-2-methyl-2-carboxyl-propylene carbonate), poly(lactide-co-hydroxybutyrate), poly(lactide-co-3-hydroxybutyrate), poly(lactide-co-4-hydroxybutyrate), poly(glycolide-co-hydroxybutyrate), poly(glycolide-co-3-hydroxybutyrate), poly(glycolide-co-4-hydroxybutyrate), poly(lactide-co-hydroxyvalerate), poly(lactide-co-3-hydroxyvalerate), poly(lactide-co-4-hydroxyvalerate), poly(glycolide-co-hydroxyvalerate), poly(glycolide-co-3-hydroxyvalerate), poly(glycolide-co-4-hydroxyvalerate), poly(3-hydroxybutyrate-co-4-hydroxybutyrate), poly(hydroxybutyrate-co-hydroxyvalerate), poly(3-hydroxybutyrate-co-3-hydroxyvalerate), poly(3-hydroxybutyrate-co-4-hydroxyvalerate), poly(4-hydroxybutyrate-co-3-hydroxyvalerate), poly(4-hydroxybutyrate-co-4-hydroxyvalerate), poly(İ-caprolactone-co-fumarate), poly(İ-caprolactone-co-propylene fumarate), poly(ester-co-ether), poly(lactide-co-ethylene glycol), poly(glycolide-co-ethylene glycol), poly(İ-caprolactone-co-ethylene glycol), poly(ester-co-amide), poly(DETOSU-1,6HD-co-DETOSU-t-CDM), poly(lactide-co-cellulose ester), poly(lactide-co-cellulose acetate), poly(lactide-co-cellulose butyrate), poly(lactide-co-cellulose acetate butyrate), poly(lactide-co-cellulose propionate), poly(glycolide-co-cellulose ester), poly(glycolide-co-cellulose acetate), poly(glycolide-co-cellulose butyrate), poly(glycolide-co-cellulose acetate butyrate), poly(glycolide-co-cellulose propionate), poly(lactide-co-glycolide-co-İ-caprolactone), poly(lactide-co-glycolide-co-trimethylene carbonate), poly(lactide-co-İ-caprolactone-co-trimethylene carbonate), poly(glycolide-co-İ-caprolactone-co-trimethylene carbonate), poly(3-hydroxybutyrate-co-3-hydroxyvalerate-co-4-hydroxybutyrate), poly(3-hydroxybutyrate-co-4-hydroxyvalerate-co-4-hydroxybutyrate), collagen, casein, polysaccharides, cellulose, cellulose esters, cellulose acetate, cellulose butyrate, cellulose acetate butyrate, cellulose propionate, chitin, chitosan, dextran, hyaluronic acid, starch, modified starch, and copolymers and blends thereof, wherein lactide includes L-lactide, D-lactide and D,L-lactide.

RESOMERS® identified by an “RG” or “DLG” in the product name, such as RG752S, is a poly(D,L-lactide-co-glycolide) or PLGA.

The synthesis of various molecular weights of DLG with various D,L-lactide-glycolide ratios is possible. In one embodiment, DLG, such as 1A, with an inherent viscosity of approximately 0.05 to approximately 0.15 dL/g can be used. In another embodiment, DLG, such as 2A, with an inherent viscosity of approximately 0.15 to approximately 0.25 dL/g can be used. Poly(D,L-lactide-co-glycolide) or PLGA copolymers can be synthesized at different ratios of lactide to glycolide, such as a lactide:glycolide ratio of 75:25. These copolymers can be an ester-terminated PLGA copolymer, as identified by the terminal “S” in the product name, or an acid-terminated PLGA copolymer, as identified by the terminal “H” in the product name.

In some embodiments, the ocular implant of the disclosure comprises at least one PLGA, wherein each PLGA is independently selected from the group consisting of RG502, RG502S, RG502H, RG503, RG503H, RG504, RG504H, RG505, RG506, RG653H, RG752H, RG752S, RG753H, RG753S, RG755, RG755S, RG756, RG756S, RG757S, RG750S, RG858, and RG858S.

In some embodiments, the ocular implant comprises from about 30 wt % to about 90 wt % polymer matrix. In some embodiments, the ocular implant comprises from about 10 wt % to about 70 wt % statin.

In some embodiments, the polymer matrix comprises poly(DL-lactide), poly(DL-lactide-co-glycolide), or a mixture thereof.

In some embodiments, the polymer matrix comprises a mixture of poly(DL-lactide) and poly(DL-lactide-co-glycolide). In some embodiments, the polymer matrix is a 50/50, 55/45, 65/35, 75/25, 85/15, 90/10, or 95/5 ratio of poly(DL-lactide)/poly(DL-lactide-co-glycolide).

In some embodiments, the polymer matrix comprises an acid end poly(DL-lactide-co-glycolide), an acid end poly(DL-lactide), or a mixture thereof. In some embodiments, the polymer matrix comprises an acid end poly(DL-lactide-co-glycolide). In some embodiments, the polymer matrix comprises an acid end poly(DL-lactide). In some embodiments, the polymer matrix comprises a mixture of acid end poly(DL-lactide-co-glycolide) and acid end poly(DL-lactide).

In some embodiments, the polymer matrix comprises an ester end poly(DL-lactide-co-glycolide), an ester end poly(DL-lactide), or a mixture thereof. In some embodiments, the polymer matrix comprises an ester end poly(DL-lactide-co-glycolide). In some embodiments, the polymer matrix comprises an ester end poly(DL-lactide). In some embodiments, the polymer matrix comprises a mixture of ester end poly(DL-lactide-co-glycolide) and ester end poly(DL-lactide).

In some embodiments, the polymer matrix has an inherent viscosity of about 0.05 to about 1.7 dL/g. In some embodiments, the polymer matrix has an inherent viscosity of about 0.5 to about 1.7 dL/g.

In some embodiments, the polymer matrix comprises poly(D, L-lactide-co-glycolide) (PLGA) having a molar ratio (D,L LA:GA) of 50:50, an inherent viscosity range (dl/g) of 0.1-0.3, 0.2-0.4, 0.4-0.6, 0.6-0.8, 0.8-1.0, 1.0-1.2, or 1.2-1.4, and acid/ester end group, such as Viatel™ DLG 5002, 5003, 5005, 5007, 5009, 5011, or 5013 A/E.

In some embodiments, the polymer matrix comprises poly(D, L-lactide-co-glycolide) (PLGA) having a molar ratio (D,L LA:GA) of 55:45, an inherent viscosity range (dl/g) of 0.2-0.4 or 0.4-0.6, and acid/ester end group, such as Viatel™ DLG 5503 or 5505 A/E.

In some embodiments, the polymer matrix comprises poly(D, L-lactide-co-glycolide) (PLGA) having a molar ratio (D,L LA:GA) of 65:35, an inherent viscosity range (dl/g) of 0.2-0.4, and acid/ester end group, such as Viatel™ DLG 6503 A/E.

In some embodiments, the polymer matrix comprises poly(D, L-lactide-co-glycolide) (PLGA) having a molar ratio (D,L LA:GA) of 75:25, an inherent viscosity range (dl/g) of 0.1-0.3, 0.2-0.4, 0.4-0.6, 0.6-0.8, 0.8-1.0, 1.0-1.2, or 1.2-1.4, and acid/ester end group, such as Viatel™ DLG 7502, 7503, 7505, 7507, 7509, 7511, or 7513 A/E.

In some embodiments, the polymer matrix comprises poly(D, L-lactide-co-glycolide) (PLGA) having a molar ratio (D,L LA:GA) of 85:15, an inherent viscosity range (dl/g) of 0.1-0.3, 0.2-0.4, 0.4-0.6, 0.6-0.8, 0.8-1.0, 1.0-1.2, or 1.2-1.4, and acid/ester end group, such as Viatel™ DLG 8502, 8503, 8505, 8507, 8509, 8511, or 8513 A/E.

In some embodiments, the polymer matrix comprises poly(D, L-lactide) (PDLLA) having an inherent viscosity range (dl/g) of 0.1-0.3, 0.2-0.4, 0.4-0.6, 0.6-0.8, 0.8-1.0, 1.0-1.2, or 1.2-1.4, and acid/ester end group, such as Viatel™ DL 02, 03, 05, 07, 09, 11, or 13 A/E.

In some embodiments, the biodegradable polymer comprises a poly(lactic-co-glycolic acid) (PLGA), wherein the PLGA is selected from the group consisting of RG502, RG503H, RG503, RG752S, RG753S, RG755S, RG756S, and RG858S. In some embodiments, the biodegradable polymer comprises a poly(lactic-co-glycolic acid) (PLGA), wherein the PLGA is selected from the group consisting of RG502, RG503, RG752S, RG753S, RG755S, RG756S, and RG858S.

In some embodiments, the ocular implant comprises one PLGA. In some embodiments, the PLGA has a ratio of PLA and PLG of about 65:35. The polymers used to form the implants of the disclosure have independent properties associated with them that when combined provide the properties needed to provide sustained release of a therapeutically effective amount of an active agent. A few of the primary polymer characteristics that control active agent release rates are the molecular weight distribution, polymer endgroup (i.e., acid or ester), and the ratio of polymers and/or copolymers in the polymer matrix. The present disclosure provides an example of a polymer matrix that possess desirable active agent release characteristics by manipulating one or more of the aforementioned properties to develop a suitable ocular implant. Exemplary formulations are shown in Table 1, below.

TABLE 1
Formulations

| | | | | Polymer |
|---|---|---|---|---|
| Group | API Form | % API | Polymer | (wt %) |
| 1 | Free Acid | 25 | 6535 PLGA Acid End-capped | 75 |
| 2 | Free acid | 40 | 7525 PLGA Ester End-capped | 60 |
| 3 | Ca²⁺ Salt | 40 | 7525 PLGA Acid End-capped | 60 |
| 4 | Ca²⁺ Salt | 40 | 7525 PLGA Ester End-capped | 60 |
| 5 | Ca²⁺ Salt | 60 | PLA Ester End capped | 40 |
| 6 | Ca²⁺ Salt | 60 | PLA Acid End capped and 6535 | 40 |
| | | | PLGA Ester end capped | |

In some embodiments, the ocular implant comprises a statin selected from the group consisting of atorvastatin, rosuvastatin, pravastatin, pitavastatin, simvastatin, lovastatin, mevastatin, and fluvastatin, or a salt thereof.

In some embodiments, provided is an ocular implant comprising a statin selected from the group consisting of atorvastatin, rosuvastatin, pravastatin, pitavastatin, simvastatin, lovastatin, mevastatin, and fluvastatin, or a salt thereof; and a polymer matrix comprising acid or ester end-capped PLA polymers or acid or ester end-capped PLGA 50/50, 65/15, 75/25, 85/15 polymers; wherein the statin is dispersed in the polymer matrix and the polymer matrix controls release of the statin.

In some embodiments, the acid or ester end-capped PLA polymers or acid or ester end-capped PLGA 50/50, 65/15, 75/25, 85/15 polymers, are selected from PLGA 5002 A, PLGA 5002 E, PLGA 7502 A, PLGA 7502 E, PLGA 8503 E, PLGA RG653 H, PLGA RG752 S, PLA DL 02 A, PLA DL 03 E, PEG 3350, PLGA 7502 A, and PLA DL 03 E, or a mixture thereof.

In some embodiments, provided is an ocular implant comprising a statin and a polymer matrix comprising PLGA 5002 A, PLGA 5002 E, PLGA 7502 A, PLGA 7502 E, PLGA 8503 E, PLGA RG653 H, PLGA RG752 S, PLA DL 02 A, PLA DL 03 E, PEG 3350, PLGA 7502 A, or PLA DL 03 E, or a mixture thereof; wherein the statin is dispersed in the polymer matrix and the polymer matrix controls release of the statin.

In some embodiments, provided is an ocular implant comprising a statin selected from the group consisting of atorvastatin, rosuvastatin, pravastatin, pitavastatin, simvastatin, lovastatin, mevastatin, and fluvastatin, or a salt thereof; and a polymer matrix comprising PLGA 5002 A, PLGA 5002 E, PLGA 7502 A, PLGA 7502 E, PLGA 8503 E, PLGA RG653 H, PLGA RG752 S, PLA DL 02 A, PLA DL 03 E, PEG 3350, PLGA 7502 A, PLA DL 03 E, or PEG 3350, or a mixture thereof; wherein the statin is dispersed in the polymer matrix and the polymer matrix controls release of the statin.

In some embodiments, provided is an ocular implant comprising a statin selected from the group consisting of atorvastatin, rosuvastatin, pravastatin, pitavastatin, simvastatin, lovastatin, mevastatin, and fluvastatin, or a salt thereof; and a polymer matrix comprising acid or ester end-capped PLA polymers or PLGA 50/50, 65/15, 75/25, 85/15 polymers, or a mixture thereof; wherein the statin is dispersed in the polymer matrix, the polymer matrix controls release of the statin, and the implant comprises from about 10 wt % to about 40 wt % statin.

In some embodiments, provided is an ocular implant comprising a statin selected from the group consisting of atorvastatin, rosuvastatin, pravastatin, pitavastatin, simvastatin, lovastatin, mevastatin, and fluvastatin, or a salt thereof; and a polymer matrix comprising PLGA 5002 A, PLGA 5002 E, PLGA 7502 A, PLGA 7502 E, PLGA 8503 E, PLGA RG653 H, PLGA RG752 S, PLA DL 02 A, PLA DL 03 E, PEG 3350, PLGA 7502 A, PLA DL 03 E, or PEG 3350, or a mixture thereof; wherein the statin is dispersed in the polymer matrix, the polymer matrix controls release of the statin, and the implant comprises from about 10 wt % to about 40 wt % statin.

In some embodiments, provided is an ocular implant comprising a statin selected from the group consisting of atorvastatin, rosuvastatin, pravastatin, pitavastatin, simvastatin, lovastatin, mevastatin, and fluvastatin, or a salt thereof; and a polymer matrix comprising acid or ester end-capped PLA polymers or PLGA 50/50, 65/15, 75/25, 85/15 polymers, or a mixture thereof; wherein the statin is dispersed in the polymer matrix, the polymer matrix controls release of the statin, and the implant comprises 10 wt %, 25 wt %, 30 wt %, or 40 wt % statin.

In some embodiments, provided is an ocular implant comprising a statin selected from the group consisting of atorvastatin, rosuvastatin, pravastatin, pitavastatin, simvastatin, lovastatin, mevastatin, and fluvastatin, or a salt thereof; and a polymer matrix comprising PLGA 5002 A, PLGA 5002 E, PLGA 7502 A, PLGA 7502 E, PLGA 8503 E, PLGA RG653 H, PLGA RG752 S, PLA DL 02 A, PLA DL 03 E, PEG 3350, PLGA 7502 A, PLA DL 03 E, or PEG 3350, or a mixture thereof; wherein the statin is dispersed in the polymer matrix, the polymer matrix controls release of the statin, and the implant comprises 10 wt %, 25 wt %, 30 wt %, or 40 wt % statin.

Suitable polymeric materials or compositions for use in the implants include those materials which are compatible, which is biocompatible, with the eye so as to cause no substantial interference with the functioning or physiology of the eye.

The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present implants.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate.

Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyvinyl alcohol, polyesters, polyethers and combinations thereof which are biocompatible and may be biodegradable and/or bioerodible.

Some exemplary characteristics of the polymers or polymeric materials for use in the present disclosure may include biocompatibility, compatibility with the therapeutic component, ease of use of the polymer in making the drug delivery systems of the present disclosure, a half-life in the physiological environment of at least about 6 hours, or greater than about one day, not significantly increasing the viscosity of the vitreous, and water insolubility.

The biodegradable polymeric materials which are included to form the matrix are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation and/or erosion of the polymer and hence the extended release profile of the implant is the relative average molecular weight of the polymeric composition employed in the implant. Different molecular weights of the same or different polymeric compositions may be included in the implant to modulate the release profile. In certain implants, the relative average molecular weight of the polymer will range from about 9 to about 64 kD, usually from about 10 to about 54 kD, and more usually from about 12 to about 45 kD.

In some implants, copolymers of glycolic acid and lactic acid are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the implant, where a more flexible implant is desirable for larger geometries. The % of polylactic acid in the polylactic acid polyglycolic acid (PLGA) copolymer can be 0-100%, about 15-85%, or about 35-65%. In some implants, a 50/50 PLGA copolymer is used.

The biodegradable polymer matrix of the ocular implant may comprise a mixture of two or more biodegradable polymers. For example, the implant may comprise a mixture of a first biodegradable polymer and a different second biodegradable polymer. One or more of the biodegradable polymers may have terminal acid groups.

In an embodiment, the ocular implants can have an aspect ratio of width-to-length from 1:1 to greater than 1:30. In some embodiments, the width-to-length aspect ratio of the ocular implant is between 1:2 to 1:25. In some embodiments, the width-to-length aspect ratio of the ocular implant is between 1:5 to 1:20. In some embodiments, the width-to-length aspect ratio of the ocular implant is between 1:10 to 1:20. In some embodiments, the width-to-length aspect ratio of the ocular implant is between 1:15 to 1:20.

In some embodiments, the implant has a diameter of about 100 μm to about 500 μm, or about 250 μm to about 400 μm, 100 μm to about 300 μm, 250 μm to about 300 μm, (e.g., about 250 μm or less, about 300 μm or less, about 325 μm or less, about 350 μm or less, about 375 μm or less, or about 400 μm or less). In some embodiments, the implant has a diameter of about 300 μm or less.

In some embodiments, the implant has a length of about 1 mm to about 10 mm, or about 2 mm to about 8 mm, or about 2 mm to about 7 mm, or about 2 mm to about 6 mm, or about 10 mm or less, or about 9 mm or less, or about 8 mm or less, or about 7 mm or less, or about 6 mm or less, or about 5 mm or less, or about 4 mm or less, or about 3 mm or less, or about 2 mm or less, or about 1 mm or less.

In some embodiments, where an initial and maintenance dose is contemplated, the implants may be different sizes. For example, the first implant may be smaller, e.g. 1 mm and the maintenance dose implant may be larger, e.g. 3-4 mm. In some embodiments, the implants may be different sizes and contain different statins. For example, a first implant may be administered comprising a statin selected from the group consisting of atorvastatin, rosuvastatin, pravastatin, pitavastatin, simvastatin, lovastatin, mevastatin, and fluvastatin, or a salt thereof; and a second implant may be administered comprising a statin selected from the group consisting of atorvastatin, rosuvastatin, pravastatin, pitavastatin, simvastatin, lovastatin, mevastatin, and fluvastatin, or a salt thereof; wherein the first implant and the second implant comprise different statins; and wherein the first implant is smaller than the second implant. In some embodiments, the polymer matrix is also different in the first implant versus the second implant.

In some embodiments, the implant is rod-shaped. In some embodiments, the term “rod-shaped,” refers to an implant that is elongated, narrow, and substantially cylindrical in form, much like a rod or a stick. In some embodiments, the implant is rod-shaped and has a diameter of about 100 μm to about 500 μm, and a length of about 2 mm to about 6 mm.

In some embodiments, the implant has a diameter of about 250 μm to about 400 μm; and a length of about 2 mm to about 6 mm. In some embodiments, the implant has a diameter of about 250 μm to about 300 μm; and a length of about 2 mm to about 6 mm.

In some embodiments, the implant has a volume of from about 0.1 mm³ to about 1 mm³. In some embodiments, the implant has a volume of about 0.1 mm³, or about 0.15 mm³, or about 0.16 mm³, or about 0.2 mm³, or about 0.25 mm³, or about 0.3 mm³, or about 0.35 mm³, or about 0.4 mm³, or about 0.45 mm³, or about 0.5 mm³, or about 0.55 mm³, or about 0.6 mm³, or about 0.65 mm³, or about 0.7 mm³, or about 0.75 mm³, or about 0.8 mm³, or about 0.85 mm³, or about 0.9 mm³, or about 0.95 mm³, or about 1 mm³.

In some embodiments, the implant is rod-shaped and has a volume of from about 0.1 mm³ to about 1 mm³. In some embodiments, the implant has a volume of about 0.1 mm³, or about 0.15 mm³, or about 0.16 mm³, or about 0.2 mm³, or about 0.25 mm³, or about 0.3 mm³, or about 0.35 mm³, or about 0.4 mm³, or about 0.45 mm³, or about 0.5 mm³, or about 0.55 mm³, or about 0.6 mm³, or about 0.65 mm³, or about 0.7 mm³, or about 0.75 mm³, or about 0.8 mm³, or about 0.85 mm³, or about 0.9 mm³, or about 0.95 mm³, or about 1 mm³.

The ocular implants disclosed herein may have a diameter sufficient for administration with a needle for administration by surgical implantation. For needle-injected implants, the implants may have any appropriate length so long as the diameter of the implant permits the implant to move through a needle. For example, implants having a length of about 6 mm to about 7 mm have been injected into an eye. The implants administered by way of a needle have a diameter that is less than the inner diameter of the needle. In some embodiments, the diameter is less than about 500 μm. The vitreous chamber in humans is able to accommodate relatively large implants of varying geometries, having lengths of, for example, 1 to 10 mm. In some embodiments, the implant is a pellet, such as a cylindrical pellet, e.g., rod-shaped.

In some embodiments, the implant may also be at least somewhat flexible so as to facilitate both insertion of the implant in the eye, such as in the vitreous, and accommodation of the implant. In some embodiments, the total weight of the implant is about 250-5,000 μg, or about 500-1,000 μg. For example, an implant may be about 500 μg, or about 1,000 μg.

Thus, implants can be prepared where the center may be of one material and the surface may have one or more layers of the same or a different composition, where the layers may be cross-linked, or of a different molecular weight, different density or porosity, or the like. For example, where it is desirable to quickly release an initial bolus of drug (e.g., a loading dose), the center may be a polylactate coated with a polylactate-polyglycolate copolymer, so as to enhance the rate of initial degradation. Alternatively, the center may be polyvinyl alcohol coated with polylactate, so that upon degradation of the polylactate exterior the center would dissolve and be rapidly washed out of the eye.

The implants may be of any geometry including fibers, sheets, films, microspheres, spheres, circular discs, plaques, rods, and the like. The upper limit for the implant size will be determined by factors such as toleration for the implant, size limitations on insertion, ease of handling, etc. Where sheets or films are employed, the sheets or films will be in the range of at least about 0.5 mm×0.5 mm, usually about 3-10 mm×5-10 mm with a thickness of about 0.1-1.0 mm for ease of handling. Where fibers are employed, the fiber diameter may be in the range of about 0.05 to 3 mm and the fiber length will generally be in the range of about 0.5-10 mm. Spheres may be in the range of about 0.5 μm to 4 mm in diameter, with comparable volumes for other shaped particles.

In some embodiments, the implant is other than spherically shaped. In some embodiments, the implant is not a nanoparticle.

The size and form of the implant can also be used to control the rate of release, period of treatment, and drug concentration at the site of implantation. Larger implants may deliver a proportionately larger dose, but depending on the surface to mass ratio, may have a slower release rate. The particular size and geometry of the implant are chosen to suit the site of implantation. The rate of statin release from an ocular implant may depend on several factors, including but not limited to, the surface area of the implant, statin content, and water solubility of the therapeutic agent, and speed of polymer degradation.

In some embodiments, the ocular implant comprises initially at least about 95% to about 99% (e.g., about 95%, about 96%, about 97%, about 98%, and about 99%) of the polymer matrix. In some embodiments, the ocular implant comprises initially at least 95% of the polymer matrix. In some embodiments, the ocular implant comprises initially at least about 80% to about 95% (e.g., about 80%, about 81%, about 82%, about 83%, about 84%, about 85%, about 86%, about 87%, about 88%, about 89%, about 90%, about 91%, about 92%, about 93%, about 94%, and about 95%) of the polymer matrix.

The proportions of the active agent component, polymer(s), and any other modifiers may be empirically determined by formulating several implants with varying proportions. A USP approved method for dissolution or release test can be used to measure the rate of release (USP 23; NF 18 (1995) pp. 1790-1798). For example, using the infinite sink method, a weighed sample of the implant is added to a measured volume of a solution containing 0.9% NaCl in water, where the solution volume will be such that the drug concentration after release is less than 5% of saturation. The mixture is maintained at 37 °C and stirred slowly to maintain the implants in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

In some embodiments, the biodegradable ocular implant is a sterile biodegradable ocular implant. As used herein, “sterile” refers to the composition meeting the requirements of sterility enforced by medicine regulatory authorities, such as the MCA in the UK or the FDA in the US. Tests are included in current versions of the compendia, such as the British Pharmacopoeia and the US Pharmacopoeia. In some embodiments, the biodegradable ocular implant is a substantially pure biodegradable ocular implant. In some embodiments, the biodegradable ocular implant is a medical-grade biodegradable ocular implant.

### Flowable Compositions

There are provided herein formulations that are of limited solubility in biological media, are biocompatible, and in some embodiments, biodegradable (referred to as “LSBB”), which may also be syringeable, for controlled and sustained release of an active agent or a combination of active agents. Solid, gel, or injectable controlled-sustained release systems can be fabricated by combining LSBB and an active agent. Systems can combine more than one biodegradable component as well as more than one active agent. Gels can be produced by vortex or mechanical mixing. Injectable formulations can be made by pre-mixing in a syringe or mixing of the LSBB and the active agent before or at the time of administration. Formulations may serve as coating for stents or other implants by, for example, dipping the stent in a liquid form of the formulation and then drying it. In another aspect, the solid form generally contains about 1% to about 60% of an LSBB, the gel form generally contains about 20% to about 80% of an LSBB, and an injectable form (which may be a gel or liquid form) generally contains about 30% to about 99.9% of an LSBB. In an aspect of the present disclosure, the excipient is also biodegradable or bioerodible.

Examples of solvents or excipients that may be useful as biocompatible, biodegradable and/or bioerodible excipients include, but are not limited to: (i) d-α-tocopherol; d,l-α-tocopherol; d-β-tocopherol; d,l-β-tocopherol; d-η-tocopherol; and d,l-η-tocopherol (including acetate, hemisuccinate, nicotinate, and succinate-PEG ester forms of each of the foregoing); tocotrienol isomers, and their esters; (ii) benzyl alcohol; (iii) benzyl benzoate; (iv) diethylene glycol dibenzoate; (v) triethylene glycol dibenzoate; (vi) dibenzoate esters of poly(oxyethylene) diols, such as those having a molecular weight of up to about 400 g/mol; (vii) propylene glycol dibenzoate; (viii) dipropylene glycol dibenzoate; (ix) tripropylene glycol dibenzoate; (x) dibenzoate esters of poly(oxypropylene) diols having a molecular weight of up to about 3000 g/mol; (xi) poly(oxypropylene) diols, such as those having a molecular weight of up to about 3000 g/mol; (xii) dimethyl sulfone; (xiii) triethyl, tripropyl, and tributyl esters of O-acetylcitrate; (xiv) triethyl, tripropyl, tributyl esters of citric acid; and (xv) liquid to semisolid polycarbonate oligomers, such as, but not limited to, those prepared by the polymerization of trimethylene carbonate (poly(1,3-propanediol carbonate)) or the ester exchange polymerization of diethylene carbonate with aliphatic diols or polyoxyalkane diols (poly(di-1,2-propylene glycol carbonate) or poly(tri-1,2-propylene glycol carbonate)).

Another example of biodegradable/biocompatible excipients useful in the present disclosure are “tocols.” Tocols refers to a family of tocopherols and tocotrienals and derivatives thereof, because tocopherols and tocotrienals are derivatives of the simplest tocopherol, 6-hydroxy-2-methyl-2-phytylchroman. Tocopherols are also known as a family of natural or synthetic compounds commonly called Vitamin E. Alpha-tocopherol is the most abundant and active form of this class of compounds. Other members of this class include β-, γ-, and δ-tocopherols and α-tocopherol derivatives such as tocopheryl acetate, succinate, nicotinate, and linoleate. Useful tocotrienols include d-δ-tocotreinols, and d-β-, d-γ-tocotrienols, and their esters.

Benzyl benzoate (CAS 120-51-4, FW 212.3) is a relatively nontoxic liquid which when applied topically in the eye results in no damage. Grant, Toxicology of the Eye 185 (2d ed., 1974). Its oral Ld50 in humans is estimated to be 0.5 g/kg-5.0 g/kg. Gosselin et al., II Clin Tox of Commercial Prod. 137 (4th ed., 1976). In vivo, benzyl benzoate is hydrolyzed to benzoic acid and benzyl alcohol. The benzyl alcohol is subsequently oxidized to benzoic acid, which is then conjugated with glucuronic acid and excreted in the urine as benzoylglucuronic acid. To a lesser extent, benzoic acid is conjugated with glycine and excreted in the urine as hippuric acid. Handbook of Pesticide Toxicology 1506 (Hayes & Laws, eds., 1991). U.S. Pat. No. 7,906,136 and Lim, Jennifer I., Marcia Niec, and Vernon Wong. “One year results of a phase 1 study of the safety and tolerability of combination therapy using sustained release intravitreal triamcinolone acetonide and ranibizumab for subfoveal neovascular AMD.” British Journal of Ophthalmology 99.5 (2015): 618-623 are incorporated herein by reference their entirety.

In addition to the active agent and the drug release sustaining component, the compositions disclosed herein may optionally include one or more buffering agents, preservatives, antioxidants, or other excipients, or combinations thereof. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents are advantageously present in amounts sufficient to maintain a pH of the system of between 2 to 9 or 4 to 8. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These buffering agents, preservatives, antioxidants, and other excipients may be present in amounts of from 0.001 to 10% by weight of the implant. Examples of antioxidant agents include ascorbate, ascorbic acid, alpha-tocopherol, mannitol, reduced glutathione, various carotenoids, cysteine, uric acid, taurine, tyrosine, superoxide dismutase, lutein, zeaxanthin, cryptoxanthin, astaxanthin, lycopene, N-acetyl-cysteine, carnosine, gamma-glutamylcysteine, quercitin, lactoferrin, dihydrolipoic acid, citrate, Ginkgo biloba extract, tea catechins, bilberry extract, vitamins E or esters of vitamin E, and retinyl palmitate.

### Methods of Treatment

The methods provided can be used to slow growth of and/or regress drusen (e.g., soft drusen), to slow growth of and/or regress drusenoid pigment epithelial detachments (PEDs), to slow and/or prevent atrophy of any or all layers of the retina (e.g. the RPE), to slow and/or prevent atrophy of one or more photoreceptors, to slow and/or prevent loss of visual function, to improve visual acuity, to prevent AMD, to slow and/or prevent progression from early AMD to intermediate AMD, to slow and/or prevent progression from intermediate AMD to Geographic Atrophy and/or to wet AMD.

Age-related changes to the retina and the choroid of the eye which contribute to and comprise the development of age-related macular degeneration (AMD) include changes in Bruch's Membrane permeability, accumulation of drusen, the loss of rod photoreceptors, the thinning of the choroid, and the accumulation of lipofuscin and reportedly components thereof (e.g., A2E (N-retinylidene-N-retinyl-ethanolamine)) in the retinal pigment epithelium (RPE) as well as lipids in the sub-RPE basal lamina (sub-RPE-BL) space and anterior to and/or within Bruch's membrane (BrM). Cholesterol rich lipoprotein particles and other constituents accumulate, forming basal linear deposits (BLinD) and drusen on the BrM. The RPE secretes apolipoproteins including but not limited to apolipoprotein B and/or E (apoB, apoE)-containing lipoprotein particles onto BrM, where they accumulate with age and eventually form a lipid-rich layer on BrM. This lipid-rich layer is frequently referred to as BLinD and/or a druse (plural drusen). Drusen negatively impact the health and function of the RPE as they inhibit nutrient exchange with the choroid and create a hypoxic environment for the highly metabolically active RPE. As the RPE is responsible for photoreceptor maintenance, the accumulation and increased thickness of drusen lead to RPE dysfunction and death, which in turn leads to death of photoreceptors and results in blindness. While anti-VEGF therapy has proven effective for treating the neovascular or “wet” form of AMD, the more common “dry” form has limited effective therapies and is a leading cause of blindness. Drusen underlie the pathogenesis of both wet and dry AMD and are thus an important target.

Drusen are extracellular deposits rich in lipids (e.g., esterifed cholesterol (EC) and phospholipids) and lipoprotein components (e.g., apoB and/or apoE) and form in the sub-RPE-BL space between the RPE-BL and the inner collagenous layer of the BrM, possibly as a result of RPE secretion of EC-rich lipoprotein particles, which resemble density lipoproteins (LDLs) and/or very low-density lipoproteins (VLDLs) basolaterally. “Hard” drusen are small, distinct and far away from one another, and may not cause vision problems for a long time, if at all. In contrast, “soft” drusen are large, have poorly defined edges, and cluster closer together. Soft drusen are more fragile than hard drusen, are oily upon dissection due to a high lipid constitution, and are a major risk factor for the development of advanced atrophic or neovascular AMD. Esterified cholesterol and phospholipids (in the form of lipoprotein particles of 50-80 nm diameter) accumulate in the BrM and the sub-RPE-BL space throughout adulthood and eventually aggregate as BLinD on the BrM or soft drusen in the sub-RPE-BL space of older eyes. Soft drusen and BLinD are two forms (a lump and a thin layer, respectively) of the same lipid-rich extracellular lesion containing lipoprotein-derived debris and specific to AMD. Lipid constituents of soft drusen and BLinD interact with reactive oxygen species to form pro-inflammatory peroxidized lipids (or lipid peroxides), which inhibit paraoxonase 1 activity, activate the complement system and elicit choroidal neovascularization. Furthermore, drusen contain immunogenic complement components. EC-rich, apoB/apoE-containing lipoproteins (e.g., LDLs, and/or LDL-like particles and/or VLDLs and/or VLDL-like particles) secreted by RPE cells are retained by a BrM that progressively thickens with age, until an oily layer forms on the BrM, with oxidation of lipids or other modifications followed by fusion of individual lipoproteins over time to form BLinD. An inflammatory response to the accumulated material ensues with activation of the complement system and other components of the immune system. Moreover, by altering the BrM with subsequent calcification and fracture, the accumulation of lipid-containing material leads to neovascularization in the sub-RPE-BL space and breakthrough to the subretinal space, the potential space between the photoreceptors and the RPE. Furthermore, the lipid-rich drusen in the sub-RPE-BL space and BLinD overlying the BrM block oxygen and nutrients (including vitamin A) from reaching the RPE cells and the photoreceptors (rods and cones) in the retina, which results in their atrophy/degeneration and eventually death.

Chronic inflammatory responses to the changes described above include complement-mediated pathways, infiltration by circulating macrophages, and activation of inflammasomes and microglia. Activation of the complement cascade leads to activation of the central component 3 (C3) and initiation of the terminal pathway with the cleavage of component 5 (C5) into C5a and C5b. The terminal pathway results in the assembly of a membrane attack complex (MAC), e.g., in the basal RPE membrane, the BrM or the choriocapillary endothelial cell membrane, by stepwise binding of C5b, C6, C7, C8 and polymerized C9 to form a pore in the lipid bilayer of the membrane. The MAC can lead to the dysfunction and death of the RPE, the BrM and/or the choriocapillary endothelium, with outer retinal atrophy ensuing. In addition, C5a elicits pro-inflammatory and pro-angiogenic effects, and combined with calcification and fracture of the BrM, can contribute to NV, including choroidal NV (CNV).

The early stage of AMD (which is atrophic AMD) is characterized by the presence of a few small to medium-size drusen and pigmentary abnormalities such as hyperpigmentation or hypopigmentation of the RPE. The intermediate stage of AMD is characterized by the presence of at least one of one large druse, multiple medium-size drusen, hyperpigmentation and/or hypopigmentation of the RPE, either without geographic atrophy (GA), or with geographic atrophy (GA) that does not extend to the center of the macula (non-foveal GA). GA represents the loss of photoreceptor, RPE and choriocapillaris, resulting in a sharply defined atrophic lesions visually resembling geographic areas on a map. In GA, RPE below the retina atrophies, which causes vision loss through the death of photoreceptors. RPE atrophy can result from a large accumulation of drusen and/or BLinD that contributes to the death of the overlying RPE, as the drusen become thick and the RPE is far removed from the choriocapillaris. Drusen may include calcification in the form of hydroxyapatite, and may progress to complete calcification, at which stage RPE cells have died. The RPE-BL thickens in a stereotypic manner to form basal laminar deposits (BLamD); RPE cells hence reside on a thick layer of BLamD. Junctions between the normally hexagonal-shaped RPE cells may be perturbed, and individual RPE cells may round up, stack and migrate anteriorly into the neurosensory retina, at which point the RPE cells become farther removed from their supply of nutrients and oxygen in the choriocapillaris. Once RPE cells begin the anterior migration, the overall RPE layer begins to atrophy.

Sub-RPE-BL drusen elevate the RPE off the BrM and thereby can cause mild vision loss, including metamorphopsia (a vision defect in which objects appear to be distorted) through disturbance of overlying photoreceptors and slowing of rod-mediated dark adaptation. Non-central GA spares the fovea and thus preserves central vision. However, patients with non-central GA can experience visual disturbances due to paracentral blind spots (scotomas), which can impair vision in dim light, decrease contrast sensitivity and impair reading ability.

The most advanced stage of nonexudative AMD is characterized by the presence of drusen and GA that extends to the center of the macula (central or subfoveal GA). Central GA involves the fovea and thus results in significant loss of central vision and visual acuity.

The advanced stage of AMD that becomes neovascular or “wet” AMD is characterized by neovascularization and any of its potential sequelae, including leakage (e.g., of plasma), plasma lipid and lipoprotein deposition, sub-RPE-BL, subretinal and intraretinal fluid, hemorrhage, fibrin, fibrovascular scars and RPE detachment. In CNV, new blood vessels grow up from the choriocapillaris and through the BrM, which causes vision loss via the aforementioned sequelae. There are three types of neovascularization (NV). Type 1 NV occurs in the sub-RPE-BL space, and new blood vessels emanate from the choroid under the macular region. Type 2 NV occurs in the subretinal space above the RPE, and new blood vessels emanate from the choroid and break through to the subretinal space. In types 1 and 2 NV, new blood vessels cross the BrM and may ramify in the pro-angiogenic cleavage plane created by soft drusen and BLinD. Type 3 NV (retinal angiomatous proliferation) occurs predominantly within the retina (intraretinal), but can also occur in the subretinal space, and new blood vessels emanate from the retina with possible anastomoses to the choroidal circulation. Type 3 NV is the most difficult subtype of NV to diagnose and has the most devastating consequences for photoreceptor health, but type 3 NV responds well to treatment with an anti-VEGF agent. A neovascular AMD patient can also have a combination of subtypes of NV, including type 1 plus type 2, type 1 plus type 3, and type 2 plus type 3. The approximate occurrence of the different subtypes of NV among newly presenting neovascular AMD patients is: 40% type 1, 9% type 2, 34% type 3, and 17% mixed (of the mixed, 80% type 1 plus type 2, 16% type 1 plus type 3, and 4% type 2 plus type 3). Another form of NV is polypoidal vasculopathy, which is of choroidal origin and is the most common form of NV among Asians, whose eyes generally have few drusen but may have BLinD. The RPE can become detached from the BrM in each subtype of NV. For instance, leakage of fluid from neovessels into the sub-RPE-BL space in type 1 NV can result in pigment epithelium detachment. The new blood vessels generated by NV are fragile, leading to leakage of fluid, blood and proteins below the macula. Leakage of blood into the subretinal space is particularly toxic to photoreceptors, and intraretinal fluid signifies a poor prognosis for vision. Bleeding and leaking from the new blood vessels, with subsequent fibrosis, can cause irreversible damage to the retina and rapid vision loss if left untreated.

In the early, intermediate and advanced stages of AMD, and in atrophic AMD and neovascular AMD, the progression and treatment of AMD can be monitored using various imaging methods known in the art (called “diagnostic” methods herein for simplicity). Such imaging methods include structural Spectral Domain Optical Coherence Tomography (SDOCT), which reveals drusen and RPE and can allow quantification of total drusen volume and monitoring the progression of the disease), color fundus photography, fundus autofluorescence (which can detect fluorophores unique to drusen and basal linear deposits), quantitative fundus autofluorescence (qAF, which relies on both blue and green autofluorescence imaging), OCT-angiography (OCT-A, which can detect the presence of sub-RPE-BL, subretinal or intraretinal fluid consistent with active neovascularization), and fluorescein angiography (which can demonstrate the types of CNV lesions). Functional measures can assess cone-mediated vision (e.g., best-corrected visual acuity [BCVA, which persists until late in the disease] on Early Treatment Diabetic Retinopathy Study (ETDRS) or Snellen charts, contrast sensitivity using a Pelli-Robson chart and other methods, low-luminance visual acuity [visual acuity measured with a neutral-density filter to reduce retinal illuminance] and rod-mediated vision (e.g., rod intercept time on dark adaptation testing, which is a sensitive measure of macular function that tracks with progression of the early disease]). For example, treatment is expected to reduce loss of and/or keep stable, and/or improve, photopic (daylight) vision mediated by cone photoreceptors and scotopic (night) vision mediated by rod photoreceptors. As another example, the loss of RPE cells can be assessed by the area of hypoautofluorescence on qAF, which can demonstrate reduced RPE area loss or stability. GA area on qAF is an FDA-approved endpoint for this stage of AMD, and has been used to monitor the progression of non-central GA or central GA and response to investigational therapies in clinical trials. The health of RPE cells can also be assessed with SDOCT. The presence of hyper-reflective foci located vertically above drusen within the retina indicates migratory RPE cells or pigmented monocytic cells and constitute a strong predictor of future atrophy of RPE cells and photoreceptors. Poor RPE health can be an indicator of poor visual outcome in both nonexudative and exudative AMD.

As with dosage per administration, total dosage over a period of about 1 month, total dosage over a period of about 6 months, total dosage for the entire treatment regimen, dosing frequency and total number of administrations, the duration/length of treatment with the active agent can be adjusted if desired and can be selected by the treating physician to minimize treatment burden and to achieve desired outcome(s), such as reduction of lipid deposits to a desired level (e.g., the presence of a few medium-size drusen or the absence of any large druse) and elimination or reduction of geographic atrophy (non-central or central) to a desired level. In some embodiments, the treatment regimen with the active agent lasts for about 24 months or less, 18 months or less, 12 months or less, or 6 months or less. In further embodiments, the treatment regimen with the active agent lasts for about 18-24 months, 12-18 months, 6-12 months, or 1-3 months. Treatment with the active agent can also last longer than 24 months (2 years), such as up to about 3 years, 4 years, 5 years or longer. In some embodiments, the treatment regimen with the active agent lasts for about 24, 21, 18, 15, 12, 9 or 6 months. In certain embodiments, the treatment regimen with the active agent lasts for about 6-12 or 12-24 months. In additional embodiments, the treatment regimen with the active agent lasts at least about 6, 12, 24 or 36 months or longer (e.g., at least about 12 months).

In some embodiments, the active agent is administered at least in the advanced stage of AMD. In certain embodiments, the active agent is administered at least in the advanced stage of AMD to treat or slow the progression of central geographic atrophy (GA), and/or to prevent or delay the onset of neovascular AMD. In further embodiments, the active agent is administered at least in the advanced stage of AMD to treat or slow the progression of neovascular AMD (including types 1, 2 and/or 3 neovascularization).

In additional embodiments, the active agent is administered at least in the intermediate stage of AMD. In certain embodiments, the active agent is administered at least in the intermediate stage of AMD to treat or slow the progression of non-central GA, and/or to prevent or delay the onset of central GA and/or neovascular AMD. In further embodiments, the active agent is administered at least in the early phase of intermediate AMD to prevent or delay the onset of non-central GA. The intermediate stage of AMD is characterized by the presence of at least one of one large druse, multiple medium-size drusen, hyperpigmentation and/or hypopigmentation of the RPE, either without geographic atrophy (GA), or with geographic atrophy (GA) that does not extend to the center of the macula (non-foveal GA). Reduction of confluent soft drusen in intermediate AMD using the active agent can result in decrease in the thickness and normalization of the Bruch's membrane, as well as renewal of the overlying RPE cell layer due to improved exchange of incoming oxygen and nutrients and outgoing waste between the choriocapillaris and the RPE. Reduction of confluent soft drusen can be observed by SDOCT.

In further embodiments, the active agent is administered at least in the early stage of AMD. The active agent can be administered at an earlier stage (e.g., the early stage or the intermediate stage) of AMD to slow or stop the progression of AMD. In some embodiments, the active agent is administered at least in the early stage of AMD to prevent or delay the onset of non-central GA. The active agent does not need to eliminate or remove all or most of the abnormal lipid deposits from the eye to have a therapeutic or prophylactic effect in AMD. If a threshold amount of abnormal lipids is cleared from the eye, natural transport mechanisms, including traffic between the choriocapillaris endothelium and the RPE layer, can properly work again and can clear remaining abnormal lipids from the eye. Furthermore, lipids accumulate in the eye slowly over a period of years (although fluctuations in druse volume in a shorter time frame are detectable).

The active agent can be administered in a stage (e.g., the early, intermediate or advanced stage) of AMD for a length of time selected by the treating physician (e.g., at least about 3 months, 6 months, 12 months, 18 months, 24 months or longer) or until the disease has been successfully treated according to selected outcome measure(s) (e.g., elimination of all or most soft drusen or reduction of soft drusen volume to a certain level).

The implants and compositions provided herein can be employed for one or more of the following: 1) reduction of drusen (including soft drusen) size (e.g., diameter or volume), number or amount (e.g., by at least about 10%, 20%, 30%, 40%, 50%, 60%, 70%, 80%, 90%, 95% or 99%); 2) prevention or resolution of drusenoid PEDs (e.g., promotion of re-attachment of the RPE-BL to the BrM ICL, or flattening of a PED or decrease in the separation/distance between the detached RPE-BL and the BrM ICL by at least about 50%, 60%, 70%, 80%, 90%, 95% or 99%); 3) enhancement of the phagocytic function (e.g., phagocytosis of drusen and other undesired matter) of RPE cells (e.g., increase in the percentage of phagocytic RPE cells by at least about 33%, 50%, 66%, 80% or 100%); 4) prevention or curtailment of atrophy and death of RPE cells and photoreceptors (e.g., reduction of the area of non-central and/or central geographic atrophy by at least about 30%, 40%, 50%, 60%, 70%, 80% or 90%); 5) prevention or forestalling of progression to or development of intermediate atrophic AMD, advanced atrophic AMD or neovascular AMD; 6) prevention or curtailment of vision loss (e.g., reduction of loss of visual acuity to no more than about 5, 4, 3, 2 or 1 letter); and 7) improvement of visual acuity (e.g., by at least about 3, 6, 9 or 12 letters).

One or more of the active agents described herein can also be used to treat other eye diseases and disorders in addition to AMD. Non-limiting examples of other eye diseases and disorders that can be treated with one or more active agents described herein include age-related macular degeneration, macular drusen (small, intermediate, large), peripheral drusen, extramacular drusen, drusenoid pigment epithelial detachment (PED), drusenoid deposits, basal laminar deposits, basal linear deposits, doyne honeycomb retinal dystrophy, Malattia Leventinese, familial dominant drusen (or autosomal dominant drusen), cuticular drusen, serous detachment of RPE, drupelets, RPE atrophy, geographic atrophy, ellipsoid zone (EZ) attenuation, EZ loss, incomplete retinal pigment epithelial and outer retinal atrophy (iRORA), complete retinal pigment epithelial and outer retinal atrophy (cRORA), nascent geographic atrophy, retinal flecks, fundus flavimaculatus, Best disease, adult-onset vitelliform macular dystrophy, Best vitelliform macular dystrophy, autosomal recessive bestrophinopathy, vitelliform material, pattern dystrophy, autosomal dominant vitreoretinochoroidopathy, BEST1 gene mutation disorders, retinal emboli (in retinal artery occlusion), retinal exudates, retinal exudates secondary to retinal microaneurysm, familial exudative vitreoretinopathy (FEVR), synchysis scintillans (cholesterolosis bulbi), neuronal ceroid lipofuscinosis, Batten's Disease, retinitis pigmentosa, Bietti's crystalline dystrophy, juvenile macular degeneration (e.g., Stargardt disease), macular telangiectasia, maculopathy (e.g., age-related maculopathy (ARM) and diabetic maculopathy (DMP) (including partial ischemic DMP)), macular edema (e.g., diabetic macular edema (DME) (including clinically significant DME, focal DME and diffuse DME), Irvine-Gass Syndrome (postoperative macular edema), and macular edema following RVO (including central RVO and branch RVO)), retinopathy (e.g., diabetic retinopathy (including in patients with DME), Purtscher's retinopathy and radiation retinopathy), retinal artery occlusion (RAO) (e.g., central and branch RAO), retinal vein occlusion (RVO) (e.g., central RVO (including central RVO with cystoid macular edema (CME)) and branch RVO (including branch RVO with CME)), glaucoma (including low-tension, normal-tension and high-tension glaucoma), ocular hypertension, retinitis (e.g., Coats' disease (exudative retinitis) or retinitis pigmentosa), chorioretinitis, choroiditis (e.g., serpiginous choroiditis), uveitis (including anterior uveitis, intermediate uveitis, posterior uveitis with or without CME, and pan-uveitis), retinal detachment (e.g., in von Hippel-Lindau disease), retinal pigment epithelium (RPE) detachment, bestrophinopathy, Doyne honeycomb/dominant drusen, and diseases associated with increased intra- or extracellular lipid storage or accumulation in addition to AMD.

The implants and compositions provided herein can provide prevention of loss or improvement in one or more of the following: metamorphopsia on Amsler grid (resolve; no distortion in straight lines from previous distortion); metamorphopsia on ForeSee Home, notal vision device (resolve; line with no distortion viewed on the device, from previous line with distortion. The trend score no longer exceeds the test score change threshold); best corrected visual acuity (BCVA) or prevention of loss of BCVA (0-100 ETDRS letters); color vision (cone contrast test 0-100% of normal, 100% being normal. Farnsworth or Lanthony D-15: confusion index 1-3, with 1 being normal and 3 abnormal, total error score 11-40, 11 being normal and 40 abnormal); visual field testing (per eye: maximum is 160° in horizontal plane and 135° in the vertical plane); scotoma/visual field loss (0 to 160° horizontal, 0 to) 135°; macular sensitivity on microperimetry testing (MAIA MP: 0-36 dB, Nidek MP: 0-20 dB); dark adaptation (rod intercept time RIT from 0 to >30 min; normal RIT is considered <6.5 min, abnormal >6.5 min); change in reading speed from baseline under standard and low luminance conditions (0-38 words/minute); contrast sensitivity (MARS chart has log scale abnormal 0 to 1.92 normal, Pelli Robson 0 abnormal to 2 normal); ellipsoid zone (EZ) attenuation or loss (0 to 28.3 mm²); full field electro-retinogram (ERG): a-wave implicit times (normal 15-16.5 Hz, abnormal >16.5 (to 19 Hz), flicker peak times (normal 29-30.5 Hz, abnormal 30.5 to 35 Hz); multifocal ERG testing: response amplitude normal 27-30 nV, abnormal <27 nV, implicit time normal <29 ms, abnormal 29-33 ms; electro-oculogram (EOG) testing (Arden ratio: abnormal 0 to 1.8, 1.8 to 2 borderline, >2 normal); Size of window defects on fluorescein angiogram (GA) (0 to 17.5 mm²); NEI-VFQ (worst 0 worst to 100 best); low luminance questionnaire (worst 0 to 100 best, abnormal <80); and functional reading independence (FRI; score 1-4).

The compositions and methods provided herein can be employed to achieve one or more of the following: decrease in number of hyper-reflective foci (1-infinity, typically 5-20 per OCT 6×6 mm volume); decrease or prevention of increase in vitelliform material height/volume (height 0-1200 mm, typically around 200-250 mm and volume 0.5 mm³); decrease or prevention of increase in drusen volume (0 to 0.03 mm³ normal, over 0.03 mm³ at high risk of late AMD; range 0-0.5 mm³; decreased unesterified cholesterol in the RPE; normalization of distribution of the esterified cholesterol from the Bruch's membrane to the photoreceptor outer segments; decreased levels of 4-hydroxy-2-nonenal (HNE) adducts (lipid peroxidation by-products) in the retina; prevention of or regression of retraction of apical microvilli of RPE cells; prevention of pseudohyopyon (clinical assessment); decrease in or prevention of increase of yellows dots/flecks, punctate white opacities (typically 0 to 100); prevention of retinal arteriolar narrowing (narrow artery <50 mm); prevention of optic nerve pallor (pallor scale 0 to 4); prevention or regression of thickening of cone outer segments (0 to 2.5 mm); decrease in hyperautofluorescent lesions (0-100); prevention or decrease of sub-RPE fibrosis; prevention of RPE atrophy (0 to 28.3 mm², measured on qAF imaging); prevention of pigment epithelial detachment (serous, vascular or drusenoid); prevention of geographic atrophy (0 to 17.5 mm², measured on qAF imaging); prevention of choroidal neovascularization (as defined on FA by CNV and OCT by subretinal/intraretinal fluid); prevention of macular holes; decrease in or prevention of subretinal hemorrhage, subretinal fluid, intraretinal fluid; decrease in or prevention of macular edema (intraretinal cysts 0 to 200); prevention of iRORA, cRORA (by case definitions), treatment of neovascular AMD, prevention of neovascular AMD, slowed progression of neovascular AMD, prevention of progression of disease, slowing progression of disease, improved visual acuity, stabilization of visual acuity, improvement of visual field as measured using Amsler grid, reduction of drusen size, reduction of drusen volume, reduction in number of drusen, elimination of drusen, improvement in ocular coherence tomography metrics, reduction and/or elimination of basal laminar deposits, reduction of anti-VEGF treatments, reduction of complement-related treatments, prevention of geographic atrophy, prevention of drusen formation, and reduction in rate of GA growth.

The implants and compositions provided herein can be employed to treat or prevent one or more of the following indications (e.g., retinal diseases and disorders): age-related macular degeneration, macular drusen (small, intermediate, large), peripheral drusen, extramacular drusen, drusenoid pigment epithelial detachment (PED), drusenoid deposits, basal laminar deposits, basal linear deposits, Doyne honeycomb retinal dystrophy (Malattia Leventinese, familial dominant drusen or autosomal dominant drusen), cuticular drusen, serous detachment of RPE, drupelets, RPE atrophy, geographic atrophy, total and partial ellipsoid zone (EZ) attenuation, EZ loss, incomplete retinal pigment epithelial and outer retinal atrophy (iRORA), complete retinal pigment epithelial and outer retinal atrophy (cRORA), nascent geographic atrophy, Stargardt disease, retinal flecks, fundus flavimaculatus, Best disease, bestrophinopathy, adult-onset vitelliform macular dystrophy, Best vitelliform macular dystrophy, autosomal recessive bestrophinopathy, vitelliform material, pattern dystrophy, autosomal dominant vitreoretinochoroidopathy, BEST1 gene mutation disorders, retinal emboli (in retinal artery occlusion), retinal exudates, Coat's disease, retinal exudates secondary to retinal microaneurysm, familial exudative vitreoretinopathy (FEVR), synchysis scintillans (cholesterolosis bulbi), neuronal ceroid lipofuscinosis, Batten's Disease, retinitis pigmentosa, and Bietti's crystalline dystrophy.

The implants and compositions of the present disclosure may also be used for treating an ocular disease is selected from the group consisting of glaucoma, diabetic retinopathy (DR), retinal vein occlusion (RVO), and retinopathy of prematurity (ROP).

In some embodiments, provided is a method of treating or preventing age-related macular degeneration (AMD), comprising administering an ultralow daily dose of statin to a patient in need thereof over the course of a treatment period.

In some embodiments, the statin is administered to an eye of the patient as an extended release unit dosage form.

In some embodiments, the ultralow daily dose of statin (per eye) is about 20 μg or less, or about 18 μg or less, or about 16 μg or less, or about 15 μg or less, or about 14 μg or less, or about 13 μg or less, or about 12 μg or less, or about 11 μg or less, or about 10 μg or less, or about 9 μg or less, or about 8 μg or less, or about 7 μg or less, or about 6 μg or less, or about 5 μg or less, or about 4 μg or less, or about 3 μg or less, or about 2 μg or less, or about 1 μg or less, or about 0.1 μg or less.

In some embodiments, the ultralow daily dose of statin (per eye) is from about 0.001 μg to about 200 μg, or from about 0.005 μg to about 100 μg, or from about 0.01 μg to about 20 μg, or from about 0.05 μg to about 10 μg, or from about 0.1 μg to about 20 μg, or from about 0.1 μg to about 10 μg.

In some embodiments, the treatment period is 1-30 days, 4-52 weeks, 1-12 months, or 1-5 years.

### Active Agent Dosing and Release Kinetics

The amount of statin loaded in the implant or composition may vary and can be adjusted based on any factor(s), such as, but not limited to, release kinetics, statin potency, desired clinical outcome, patient needs, etc.

In some embodiments, the implant or composition as disclosed herein administered (per eye) comprises a total amount of statin in a range of about 0.1 μg to about 10 mg, 0.1 μg to about 5 mg, 0.1 μg to about 2 mg, 0.1 μg to about 1.5 mg, or 1 μg to about 1 mg (e.g., about 1 μg, about 10 μg, about 25 μg, about 50 μg, about 75 μg, about 100 μg, about 125 μg, about 150 μg, about 175 μg, about 200 μg, about 225 μg, about 250 μg, about 275 μg, about 300 μg, about 325 μg, about 350 μg, about 375 μg, about 400 μg, about 425 μg, about 450 μg, about 475 μg, about 500 μg, about 525 μg, about 550 μg, about 575 μg, about 600 μg, about 625 μg, about 650 μg, about 675 μg, about 700 μg, about 725 μg, about 750 μg, about 775 μg, about 800 μg, about 825 μg, about 850 μg, about 875 μg, about 900 μg, about 925 μg, about 950 μg, and about 975 μg).

In some embodiments, the implant or composition as disclosed herein comprises a dose of an active agent in a range of about 10 μg to about 500 μg.

In some embodiments, the implant or composition as disclosed herein comprises about 500 μg to about 4 mg (e.g., about 1 mg, about 1.5 mg, about 2 mg, about 2.5 mg, about 3 mg, and about 3.5 mg) of statin. In some embodiments, the implant or composition comprises about 150 μg to about 250 μg of statin.

In certain embodiments, the implant or composition as disclosed herein comprises about 165 μg to about 220 μg (e.g., about 165 μg, about 170 μg, about 175 μg, about 180 μg, about 185 μg, about 190 μg, about 195 μg, about 200 μg, about 205 μg, about 210 μg, about 215 μg, and about 220 μg) of statin. In some embodiments, the dose is about 300 μg to about 500 μg of statin. In some embodiments, the dose is about 400 μg to about 500 μg of statin. In some embodiments, the dose is about 300 μg to about 550 μg of statin. In some embodiments, the dose is about 300 μg to about 600 μg of statin.

In certain embodiments, the implant or composition as disclosed herein comprises about 330 μg to about 500 μg (e.g., about 330 μg, about 335 μg, about 340 μg, about 345 μg, about 350 μg, about 355 μg, about 360 μg, about 365 μg, about 370 μg, about 375 μg, about 380 μg, about 385 μg, about 390 μg, about 395 μg, about 400 μg, about 405 μg, about 410 μg, about 415 μg, about 420 μg, about 425 μg, about 430 μg, about 435 μg, about 440 μg, about 445 μg, about 450 μg, about 455 μg, about 460 μg, about 465 μg, about 470 μg, about 475 μg, about 480 μg, about 485 μg, about 490 μg, about 495 μg, and about 500 μg) of statin.

In some embodiments, the implant or composition as disclosed herein comprises about 200 μg to about 400 μg (e.g., about 200 μg, about 210 μg, about 220 μg, about 230 μg, about 240 μg, about 250 μg, about 260 μg, about 270 μg, about 280 μg, about 290 μg, about 300 μg, about 310 μg, about 320 μg, about 330 μg, about 340 μg, about 350 μg, about 360 μg, about 370 μg, about 380 μg, about 390 μg, about 400 μg) of statin.

In some embodiments, the implant or composition as disclosed herein comprises about 175 μg of statin. In some embodiments, when the statin is present as a salt, the amount of statin is calculated based on the molecular weight of the compound perse (e.g., the free acid or free base).

In some embodiments, the statin is atorvastatin, or a salt thereof. In some embodiments, the statin is atorvastatin free base or atorvastatin calcium salt.

Exemplary implants are described in Table 2, below.

TABLE 2
Formulations

| Statin | Polymer Matrix | Implant Weight | Total Statin in Implant |
|---|---|---|---|
| (wt %) | (wt %) | (μg) | (μg) |
| 5 | 95 | 50 | 2.5 |
| 5 | 95 | 1000 | 50 |
| 40 | 60 | 50 | 20 |
| 40 | 60 | 1000 | 400 |
| 70 | 30 | 50 | 35 |
| 70 | 30 | 1000 | 700 |

It will be understood to one of skill in the art that the weight percent (wt %) of statin can be adjusted to provide the desired release profile using methods known in the art. For example, such calculations typically take into account one or more known or experimentally determinable factors, such as, but not limited to, molecular weight, activity, efficacy, and/or half-life of the statin, degradation rate of the polymer matrix or flowable composition, release rate of statin from the polymer matrix or flowable composition (e.g., swellability, hydrophilicity or hydrophobicity), and the like.

The methods provided herein can comprise administering an implant or composition as disclosed herein to the patient once weekly, once every two weeks, once every four weeks, once every six weeks, once every two months, once every three months, once every four months, once every five months, once every six months, once every seven months, once every eight months, once every nine months, once every ten months, once every eleven months, once every twelve months, once every eighteen months, once every two years, once every three years, once every four years, once every five years, once every six years, once every seven years, once every eight years, once every nine years, once every ten years, or a number or a range between any two of the values.

In certain embodiments, the amount of statin released during the first hours or days after administration is higher than the target daily dose (e.g., during the initial burst release period) and as the implant or composition is substantially degraded and the concentration of statin in the remaining portion of the implant or composition is diminished, the daily dose will be lower than the target daily dose.

In certain embodiments, the total target daily dose (per eye) is from about 0.03 μg/mL to 30 μg/mL (mass of statin per mL of vitreous fluid), or from about 0.05 μg/mL to 20 μg/mL, or from about 0.1 μg/mL to 10 μg/mL, or from about 0.2 μg/mL to 5 μg/mL, or from about 0.3 μg/mL to 3 μg/mL per eye per day. In certain embodiments, the total target daily dose (per eye) is from about 0.3 μg/mL to 3 μg/mL (mass of statin per mL of vitreous fluid).

In certain embodiments, the target amount of statin maintained in the eye for the duration of the treatment period ranges from about 0.01 μg/mL to 1 μg/mL (mass of statin per mL of vitreous fluid). In certain embodiments, the target amount of statin maintained in the eye for the duration of the treatment period is about 0.01 μg/mL, or about 0.02 μg/mL, or about 0.03 μg/mL, or about 0.04 μg/mL, or about 0.05 μg/mL, or about 0.06 μg/mL, or about 0.07 μg/mL, or about 0.08 μg/mL, or about 0.09 μg/mL, or about 1 μg/mL (mass of statin per mL of vitreous fluid).

It will be understood that each eye may be treated independently in that the same dose or treatment may not be administered to both eyes of the patient.

In certain embodiments, the active agent comprises as a % w/w of the implant or composition as disclosed herein: about 1% to about 90%, or about 1% to about 80%, or about 1% to about 70%, or about 1% to about 60%, or about 1% to about 55%, or about 1% to about 50%, or about 1% to about 45%, or about 1% to about 40%, or about 1% to about 35%, or about 1% to about 30%, or about 1% to about 25%, or about 1% to about 20%, or about 1% to about 15%, or about 1% to about 10%, or about 1% to about 5%, or about 5% to about 90%, or about 5% to about 80%, or about 5% to about 70%, or about 5% to about 60%, or about 5% to about 55%, or about 5% to about 50%, or about 5% to about 45%, or about 5% to about 40%, or about 5% to about 35%, or about 5% to about 30%, or about 5% to about 25%, or about 5% to about 20%, or about 5% to about 15%, or about 5% to about 10%, or about 10% to about 90%, or about 10% to about 80%, or about 10% to about 70%, or about 10% to about 60%, or about 10% to about 55%, or about 10% to about 50%, or about 10% to about 45%, or about 10% to about 40%, or about 10% to about 35%, or about 10% to about 30%, or about 10% to about 25%, or about 10% to about 20%, or about 10% to about 15%, or about 15% to about 90%, or about 15% to about 80%, or about 15% to about 70%, or about 15% to about 60%, or about 15% to about 55%, or about 15% to about 50%, or about 15% to about 45%, or about 15% to about 40%, or about 15% to about 35%, or about 15% to about 30%, or about 15% to about 25%, or about 15% to about 20%, or about 20% to about 90%, or about 20% to about 80%, or about 20% to about 70%, or about 20% to about 60%, or about 20% to about 55%, or about 20% to about 50%, or about 20% to about 45%, or about 20% to about 40%, or about 20% to about 35%, or about 20% to about 30%, or about 20% to about 25%, or about 30% to about 90%, or about 30% to about 80%, or about 30% to about 70%, or about 30% to about 60%, or about 30% to about 55%, or about 30% to about 50%, or about 30% to about 45%, or about 30% to about 40%, or about 30% to about 35%, or about 40% to about 90%, or about 40% to about 80%, or about 40% to about 70%, or about 40% to about 60%, or about 40% to about 55%, or about 40% to about 50%, or about 40% to about 45%, or about 45% to about 90%, or about 45% to about 80%, or about 45% to about 75%, or about 45% to about 70%, or about 45% to about 65%, or about 45% to about 60%, or about 45% to about 55%, or about 45% to about 50%, or about 50% to about 90%, or about 50% to about 80%, or about 50% to about 70%, or about 50% to about 60%, or about 50% to about 55%, or about 25% to about 40%, or about 28% to about 35%, or about 30%, to about 33%, or about 39% to about 45%.

In certain embodiments, the implant or composition as disclosed herein administers a daily dose of statin in an amount of from about 1 μg to about 1000 μg; or about 1 μg to about 500 μg; or about 1 μg to about 400 μg; or about 1 μg to about 300 μg; or about 1 μg to about 200 μg; or about 1 μg to about 100 μg; or about 1 μg to about 90 μg; or about 1 μg to about 80 μg; or about 1 μg to about 70 μg; or about 1 μg to about 60 μg; or about 1 μg to about 50 μg; or about 1 μg to about 40 μg; or about 1 μg to about 30 μg; or about 1 μg to about 20 μg; or about 1 μg to about 10 μg or about 10 μg to about 100 μg; or about 10 μg to about 50 μg; or about 10 μg to about 35 μg; or about 10 μg to about 31 μg; or about 14 μg to about 26 μg; or about 20 μg to about 40 μg; or about 25 μg to about 35 μg; or about 28 μg to about 31 μg; or about 14 μg; or about 19 μg; or about 26 μg; or about 29 μg; or about 42 μg.

In certain embodiments, the polymer matrix or solvent comprises as a % w/w of the overall implant composition: about 5% to about 95% w/w, or about bout 5% to about 90% w/w, or about 5% to about 80%, or about 5% to about 70%, or about 5% to about 60%, or about 10% to about 90% w/w, or about 10% to about 80%, or about 10% to about 70%, or about 10% to about 60%, or about 20% to about 90%, or about 20% to about 80%, or about 20% to about 70%, or about 20% to about 60%, or about 30% to about 90%, or about 30% to about 80%, or about 30% to about 70%, or about 30% to about 60%, or about 40% to about 90%, or about 40% to about 80%, or about 40% to about 70%, or about 40% to about 60%, or about 50% to about 90%, or about 50% to about 80%, or about 50% to about 70%, or about 50% to about 60%, or about 60% to about 90%, or about 60% to about 85%, or about 65% to about 85%, or about 60% to about 80%, or about 60% to about 70%; or about 45% to about 80%, or about 45% to about 75%, or about 45% to about 70%, or about 45% to about 65%, or about 45% to about 60%, or about 45% to about 55%, or about 45% to about 50%, or about 70% to about 80%, or about 65% to about 85%, or about 85% to about 95%, or about 92.5% to about 95%, or about 55% to about 70% w/w of the implant composition.

Release of the active agent from a polymer matrix or solvent may be a function of several processes, including diffusion out of the polymer, degradation of the polymer and/or erosion or degradation of the polymer. Some factors which influence the release kinetics of active agent from the implant or composition can include the size and shape of the implant or a depot of the composition, the size of the active agent particles, the solubility of the active agent, the ratio of active agent to polymer(s) or solvent(s), the method of manufacture, the surface area exposed, and the erosion rate of the polymer(s) or solvent(s). For example, degradation or erosion by hydrolysis (among other mechanisms) can occur, and therefore, any change in the composition of the implant that enhances water uptake by the implant will likely increase the rate of hydrolysis, thereby increasing the rate of polymer degradation and erosion, and thus, increasing the rate of active agent release. Equally important to controlling the biodegradation, and hence the extended release profile of the implant, is the relative average molecular weight of the polymeric composition employed in the implant. Different molecular weights of the same or different polymers may be included in an implant to modulate the release profile.

The release kinetics of the implant or composition described herein can be dependent in part on the surface area implant or composition administered. A larger surface area may expose more active agent to ocular fluid, and may cause faster erosion of the implant or composition and dissolution of the active agent in the fluid. Therefore, the size and shape of an implant may also be used to control the rate of release, period of treatment, and active agent concentration at the site of implantation.

As discussed herein, the polymer matrix of the ocular implant may swell or degrade at a rate effective to sustain release of an effective amount of statin for weeks, months, or years after implantation into an eye. For a homopolymer such as PLA, the drug release is also determined by (a) the lactide stereoisomeric composition (i.e., the amount of L-vs. D,L-lactide) and (b) molecular weight. Three additional factors that determine the degradation rate of PLGA copolymers are: (a) the lactide:glycolide ratio, (b) the lactide stereoisomeric composition (i.e., the amount of L-vs. DL-lactide), and (c) molecular weight. The lactide:glycolide ratio and stereoisomeric composition are generally considered most important for PLGA degradation, as they determine polymer hydrophilicity and crystallinity. For instance, PLGA with a 1:1 ratio of lactic acid to glycolic acid degrades faster than PLA or PGA, and the degradation rate can be decreased by increasing the content of either lactide or glycolide. Polymers with degradation times ranging from weeks to years can be manufactured simply by customizing the lactide:glycolide ratio and lactide stereoisomeric composition.

The release rate of an active agent from an implant may be empirically determined using a variety methods. A USP approved method for dissolution or release test can be used to measure the rate of release (USP 23; NF 18 (1995) pp. 1790-1798). For example, using the infinite sink method, a weighed sample of the drug delivery system (e.g., implant) is added to a measured volume of a solution containing 0.9% NaCl in water (or other appropriate release medium such as phosphate buffered saline), where the solution volume will be such that the drug concentration after release is less than 20%, or less than 5%, of saturation. The mixture is maintained at 37 °C and stirred slowly to ensure drug release. The amount of drug released in to the medium as a function of time may be quantified by various methods known in the art, such as spectrophotometrically, by HPLC, mass spectroscopy, etc.

In some embodiments, greater than about 95%, or greater than about 90%, or greater than about 85%, or greater than about 80% of the statin is released from the ocular implant when placed in phosphate buffered saline (PBS) in about 1 month to about 12 months (about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months).

In some embodiments, less than 90% (e.g., about 90%, about 85%, about 80%, about 75%, about 70%, about 65%, about 60%, about 55%, about 50%, about 45%, about 40%, about 35%, about 30%, about 25%, about 20%, about 15%, about 10%, and about 5%) of the statin is released from the ocular implant when placed in phosphate buffered saline (PBS) in about 1 month to about 12 months (about 1 month, about 2 months, about 3 months, about 4 months, about 5 months, about 6 months, about 7 months, about 8 months, about 9 months, about 10 months, about 11 months, about 12 months).

Over the course of treatment, the biodegradable polymer matrix degrades releasing the active agent. Once the active agent has been completely released, the polymer matrix is expected to be gone. Complete polymer matrix degradation may take longer than the complete release of the active agent. Polymer matrix degradation may occur at the same rate as the release of the active agent. For example, the ocular implant may be designed to release an effective amount of active agent for approximately one month, two months, three months, four months, five months, six months, seven months, eight months, nine months, ten months, eleven months, twelve months, or longer. In aspects, the ocular implant is designed to release an effective amount of active agent for one month, two months, three months, four months, five months, or six months. In other aspects, the ocular implant is designed to release an effective amount of active agent for three months, four months, five months, or six months. In aspects, the ocular implant releases active agent for longer than 6 months. In aspects, the ocular implant releases active agent for a period of time between about 6 months and one year.

The therapeutically effective amount and the frequency of administration of, and the duration of treatment with, a particular active agent for the treatment of AMD or another eye disorder may depend on various factors, including the eye disease, the severity of the disease, the potency of the active agent, the mode of administration, the age, body weight, general health, gender and diet of the subject, and the response of the subject to the treatment, and can be determined by the treating physician. In some embodiments, the dosing regimen of one or more, or all, of the active agent(s) comprises one or more loading doses followed by one or more maintenance doses. The one or more loading doses are designed to establish a relatively high or therapeutically effective level of the active agent at the target site(s) relatively quickly, and the one or more maintenance doses are designed to establish a therapeutically effective level of the active agent for the period of treatment. The loading dose can be provided, e.g., by administering a dose that is greater than (e.g., 2, 3, 4 or 5 times greater than) the maintenance dose, or by administering a dose substantially similar to the maintenance dose more frequently (e.g., 2, 3, 4 or 5 times more frequently) at the beginning of treatment.

### Administration and Dosing

In some embodiments, the ocular implant is sized for implantation in an ocular region. The ocular implant can be configured for local, e.g., ophthalmic, administration. Local administration of an active agent can deliver the agent to the target site(s) more effectively, avoid first-pass metabolism and require a lower administration dose of the agent, and thereby can reduce any side effect caused by the agent. As the pathological events of AMD occur in the eye, the active agent(s) used to treat AMD can be locally administered to the eye for more effective treatment. Potential routes/modes of local administration include without limitation topical, intraaqueous (the aqueous humor), peribulbar, retrobulbar, suprachoroidal, subconjunctival, intraocular, periocular, subretinal, intrascleral, posterior juxtascleral, trans-scleral, sub-Tenon's, intravitreal and/or transvitreal. Subretinal administration administers an active agent below the retina, such as, e.g., the subretinal space, the RPE, the sub-RPE-BL space or the choroid, or any combination or all thereof. Potential sites of local administration include, but are not limited to, the anterior chamber (aqueous humor) and the posterior chamber of the eye, the vitreous humor (vitreous body), the retina (including the macula and/or the photoreceptor layer), the subretinal space, the RPE, the sub-RPE-BL space, the choroid (including the BrM and the choriocapillaris endothelium), the sclera, and the sub-Tenon's capsule/space. In some embodiments, an active agent is delivered across the sclera and the choroid to the vitreous humor, from where it can diffuse to the target tissue(s), e.g., the retina (e.g., photoreceptors), the subretinal space, the RPE, the sub-RPE-BL space or the BrM, or any combination or all thereof. In other embodiments, an active agent is delivered across the sclera and the choroid to the target tissue(s), e.g., the retina (e.g., photoreceptors), the subretinal space, the RPE and/or the sub-RPE-BL space, from where it can diffuse to the BrM if the BrM is a target tissue. In further embodiments, an active agent is administered intraocularly into the anterior or posterior chamber of the eye, the vitreous humor, the retina or the subretinal space, for example.

The biodegradable implants may be inserted into the eye by a variety of methods, including placement by forceps, by trocar, or by other types of applicators, after making an incision in the sclera. In some instances, a trocar or applicator may be used without creating an incision. In various embodiments, the implant is administered as an intravitreal administration. An intravitreal administration refers to drug administration into the vitreous humor of the eye. In some embodiments, the implant is administered locally to the back of the eye. In some embodiments, the implant is injected into the intravitreal space using a needle and applicator. Delivery of such implants disclosed herein include delivery through a 20 gauge needle or smaller diameter needle. In aspects, the needles can be thin-walled or ultra-thin walled. In one embodied delivery method the needle is a 20 gauge, 21 gauge, 22 gauge, 23 gauge, 24 gauge, 25 gauge, 26 gauge, 27 gauge, 28 gauge, 29 gauge, 30 gauge, 31 gauge, 32 gauge, 33 gauge, or 34 gauge needle. In aspects, the needles can be thin-walled or ultra-thin walled.

The implants of the present disclosure may be inserted into the eye by a variety of methods using a suitable ocular implant delivery device. One example may include the device disclosed in U.S. Pat. No. 6,899,717, the relevant disclosure of which is herein incorporated by reference. In one embodiment, the implant is placed in the eye(s) using an intraocular delivery apparatus, the apparatus comprising an elongate housing and a cannula extending longitudinally from the housing, the cannula having a proximal end and a distal sharp end and having a lumen extending therethrough, the lumen having an inner diameter sufficient to receive the implant and permit passage of the implant through the lumen and into the eye of the patient. The apparatus may further comprise a push rod or plunger operably connected with a user-actuated linkage for ejecting the implant through the lumen into the eye. Another embodiment of the present disclosure is an apparatus for delivering a biodegradable ocular implant into the eye of a patient, the apparatus comprising an ocular implant according to any of those described herein, an elongate housing and a cannula extending longitudinally from the housing, the cannula having a proximal end, a distal sharp end, and a lumen extending therethrough, the lumen having an inner diameter sufficient to receive the ocular implant and permit translation of the implant through the lumen and into the eye of the patient. The cannula may be a 20 gauge, 21 gauge, 22 gauge, 23 gauge, 24 gauge, 25 gauge, 26 gauge, 27 gauge, 28 gauge, 29 gauge, 30 gauge, 31 gauge, 32 gauge, 33 gauge, or 34 gauge needle, or may otherwise be described as having inner and outer diameters equivalent to those of a 20 gauge, 21 gauge, 22 gauge, 23 gauge, 24 gauge, 25 gauge, 26 gauge, 27 gauge, 28 gauge, 29 gauge, 30 gauge, 31 gauge, 32 gauge, 33 gauge, or 34 gauge needle. The needle, in addition, may be a thin-wall or ultra-thin-wall needle.

For placement e.g. in the vitreous cavity of the eye, useful implantation methods include advancing the needle through the pars plana at a location approximately 3.5-4 mm from the limbus of the eye. For smaller diameter needles, e.g., 25 gauge or smaller diameter needle, the needle can be inserted from any angle relative to the eye and still produce acceptable self-sealing results. For larger gauge needles, e.g., 23 gauge and above, self-sealing results can be enhanced by inserting the needle at angle relative to the eye surface. For example, good results are achieved by inserting the angle at an angle of 45° or less relative to the eye surface. Also, slightly improved results can be seen in some cases by orienting the bevel of the needle downward with respect to the eye surface. Another advantageous method involves a so-called “tunnel technique” approach. In this technique, the patient's eye is restrained from moving using e.g. a cotton swab or forceps, and the needle is advanced into the sclera at an angle approaching parallel relative to the eye surface. In this technique, the bevel will usually be oriented upward with respect to the eye surface. Once the tip is advanced sufficiently far enough into the scleral layer, usually such that the bevel portion is at least disposed within the scleral layer, the angle of the needle is adjusted to a more downward angle into the eye, and the needle is further advanced.

### Methods of Preparation

Various methods may be used to produce the implants. Methods include, but are not limited to, solvent casting, phase separation, interfacial methods, molding, compression molding, injection molding, extrusion, co-extrusion, heat extrusion, die cutting, heat compression, and combinations thereof. In certain embodiments, the implants are molded, such as in polymeric molds. Useful techniques include extrusion methods (for example, hot melt extrusion), compression methods, pellet pressing, solvent casting, print technology, hot embossing, soft lithography molding methods, injection molding methods, heat press methods and the like. As previously discussed, an ocular implant according to this disclosure may be configured as a rod, wafer, sheet, film, or compressed tablet. Cast films or sheets can be ground into microparticles, which may be useful in some applications. Biodegradable microspheres formed by an emulsion method and having any of the formulations described herein may also find use in a method according to this disclosure.

In some embodiments, the ocular implant of this disclosure is a solid rod-shaped implant formed by an extrusion process (an extruded rod) and is sized for placement in the anterior chamber of the eye. Methods for making an ocular implant by an extrusion process are familiar to those of skill in the art. See, for example, US 2008/0145403 and US 2005/0244464. An extruded implant (e.g., an extruded rod) can be made by a single or double extrusion method. Choice of technique, and manipulation of technique parameters employed to produce the implants can influence the release rates of the drug. Room temperature compression methods may result in an implant with discrete microparticles of drug and polymer interspersed. Extrusion methods may result in implants with a progressively more homogenous dispersion of the drug within a continuous polymer matrix, as the production temperature is increased. The use of extrusion methods may allow for large-scale manufacture of implants and result in implants with a homogeneous dispersion of the drug within the polymer matrix.

The use of extrusion methods allows for large-scale manufacture of implants and results in implants with a homogeneous dispersion of the drug within the polymer matrix. When using extrusion methods, the polymers and active agents that are chosen are stable at temperatures required for manufacturing, usually at least about 50 °C. Extrusion methods use temperatures of about 25 °C to about 150 °C, or about 60 °C to about 130 °C. The temperature used during an extrusion method should be high enough to soften the polymer but low enough to avoid substantial loss of active agent activity. In this regard, extrusion methods may use temperatures of 50 °C to 130 °C, or an extrusion temperature of between 50 °C and 80 °C, or from 55 °C to 70 °C. For example, the extrusion temperature used to make an active agent-containing implant may be 60 °C to 75 °C, or from 60 °C to 70 °C. Low temperatures such as these may be used for some active agents to preserve potency through to the final extruded implant.

Different extrusion methods may yield implants with different characteristics, including but not limited to the homogeneity of the dispersion of the active agent within the polymer matrix. For example, using a piston extruder, a single screw extruder, and a twin screw extruder may produce implants with progressively more homogeneous dispersion of the active agent. When using one extrusion method, extrusion parameters such as temperature, feeding rate, circulation time, pull rate (if any), extrusion speed, die geometry, and die surface finish will have an effect on the release profile of the implants produced.

In one variation of producing implants by a piston or twin-screw extrusion methods, the drug and polymers, including any polyethylene glycol if called for, are first mixed at room temperature and then heated to an appropriate temperature to soften the mixture or transform the mixture to a semi-molten state for a time period of 0 to 1 hour, for 1 to 10 minutes, 1 minute to 30 minutes, 1-5 minutes, 5 minutes to 15 minutes, or 10 minutes. The implants are then extruded at a temperature of between 50 °C and 80 °C. In some variations, the temperature of extrusion may range from 60-75 °C, or from 60-65 °C. In some screw extrusion methods, the powder blend of active agent and polymer is added to a single or twin screw extruder preset at a temperature of 50 °C to 130 °C, and directly extruded as a filament or rod with minimal residence time in the extruder. The extruded filament is then cut to a length suitable for placement in the anterior chamber or vitreous of the eye. The total weight of the implant will of course be proportional to the length and diameter of the implant, and implants may be cut to a desired target weight and therefore dosage of the active agent. For example, an intracameral implant in accordance with this disclosure may be cut to a target weight of between 20 and 150 μg (+5%). In some embodiments, the implants are cut to a target weight of 50 μg (+5%), 75 μg (+5%), or 100 μg (+5%), wherein 20% of the implant by weight is active agent.

In one embodiment, the method for making the implants involves dissolving the appropriate polymers and active agent in a solvent. Solvent selection will depend on the polymers and active agents chosen. For the implants described herein, dichloromethane (DCM) can be an appropriate solvent. Other solvents may include methylene chloride and ethyl acetate. Once the polymers and active agent(s) have been dissolved, the resulting mixture is cast into a die of an appropriate shape. Once cast, the solvent used to dissolve the polymers and active agent(s) is evaporated at a temperature between 20 °C and 30 °C, or about 25 °C. The polymer can be dried at room temperature or even in a vacuum. For example, the cast polymers including active agents can be dried by evaporation in a vacuum. Once the cast polymers are dried, they can be processed into an implant using any method known in the art to do so. In an example embodiment, the dried casted polymer can be cut and/or ground into small pieces or particles and extruded into rounded or squared rod shaped structures at a temperature between 50 °C and 80 °C.

Implants that are compatible with loading and ejection from apparatus according to the present disclosure can be formed by a number of known methods, including phase separation methods, interfacial methods, extrusion methods, compression methods, molding methods, injection molding methods, heat press methods and the like. Particular methods used can be chosen, and technique parameters varied, based on desired implant size and drug release characteristics.

In manufacturing an implant, it may be desirable to pre-load the implant into the cannula. Pre-loaded apparatus provide added convenience for the user and avoid unnecessary handling of implants. Further, such loading can be done under sterile conditions, thereby ensuring delivery of a sterilized implant. In some embodiments, the implant can be pre-loaded into the cannula assembly and the loaded cannula assembly incorporate into the nose cone. In this fashion, loaded nose cone/cannula assemblies can be pre-assembled, for later incorporation with the housing assembly. In some embodiments, the implant can be preloaded in the cannula and then later assembled onto the housing assembly. In an alternative variation on this embodiment, the cannula can have two separate parts, with one part of the cannula retained within the housing that then communicates with the other external portion of the cannula that is subsequently connected to the housing. In such a variation, an implant can further be preloaded in the cannula part retained within the housing. In any case, push rods and linkages of the appropriate lengths are provided dependent on the length of the particular loaded implant, such that complete ejection of the particular implant can be assured.

Label plates, or other locations on the housing, can include the appropriate information relative to particular implant loaded. Given this interchangeability, unique apparatus for the delivery of selected implants can be easily manufactured, simply by providing the particular cannula, plunger, and linkage system for the selected implant. The remaining components of the apparatus remain the same. The name plate or housing itself can be labeled to correspond to the selected implant, thus identifying the apparatus with the loaded implant.

When the apparatus is assembled with the implant pre-loaded, it may further be desirable that the implant be positioned just proximal of the opening at the cannula tip. In this fashion, the introduction of air into the eye can be avoided when the implant is ejected, as could otherwise occur where the implant located further within the cannula lumen and an air bubble or air pocket allowed to exist between the cannula tip and the implant and ejection of the implant were to force the air bubble or air pocket into the eye. One method to accomplish this is to load the implant distally into the cannula followed by the plunger, with the plunger length designed to push the implant to the desired pre-actuation position. When the cannula assembly is then installed onto the housing, the plunger and thus the implant is advanced to the desired position. To guard against inadvertent premature release of the implant, the cannula can have a slight bend incorporated into the tip such that enough friction exists between the inner wall of the cannula and the implant to hold the implant in place, but at the same time, the frictional force is easily overcome by action of the plunger to eject the implant upon actuation of the apparatus.

Other cannula designs can likewise achieve the desired effect of avoiding the introduction of air into the eye upon ejection of the implant. For example, the implant can be positioned proximally of the cannula tip but with sufficient tolerance between the implant and cannula wall to provide for air exhaust past the implant as it is moved through the cannula. Adequate tolerances are those that retain air in front of the implant at close to ambient pressure as the implant is moved along the cannula. Because fluid pressure within the eye is typically slightly positive relative to ambient pressure, air at ambient pressure will not enter the eye.

Loaded apparatus according to the disclosure can be packaged to include a safety cap extending over the cannula and securing to the housing. This will provide a measure of safety during handling of the apparatus. The button or other depression mechanism of the apparatus can also include a notch which receives the rim of the safety cap. In this configuration, the safety cap will then also operate to guard against unintentional depression of the button or other depression mechanism and ejection of the implant.

As can be appreciated, an implant delivery apparatus according to the disclosure that is provided loaded with the desired implant is of great benefit to the physician user. Such apparatus can be provided sterile packaged for a single use application. The user need not ever handle the implant itself. As previously mentioned, the apparatus provides for a controlled ejection of the implant. The configuration and design of the apparatus also helps to achieve uniform placement of implants from patient to patient. Further, when the apparatus is configured to deliver a micro-implant, the apparatus provides a self-sealing method for delivery, as previously discussed. This has enormous benefit to the physician and patient in that the entire implant procedure can safely, easily, and economically be performed in a physician's office, without the need for more costly surgical support currently required for implant delivery.

In embodiments, the implant and delivery device may be combined and presented as a kit for use. The implant may be packaged separately from the delivery device and loaded into the delivery device just prior to use. Also, the implant may be loaded into the delivery device prior to packaging. In this case, once the kit is opened, the delivery device is ready for use. Components may be sterilized individually and combined into a kit, or may be sterilized after being combined into a kit. In some embodiments, the ocular implant is a sterile ocular implant. As used herein, “sterile” refers to the composition meeting the requirements of sterility enforced by medicine regulatory authorities, such as the MCA in the UK or the FDA in the US. Tests are included in current versions of the compendia, such as the British Pharmacopoeia and the US Pharmacopoeia. In some embodiments, the ocular implant is a substantially pure ocular implant. In some embodiments, the ocular implant is a medical-grade ocular implant. In some embodiments, the ocular implant is administered into the intravitreal space every 3 to 12 months.

### EXAMPLES

### Example 1: Atorvastatin Bioresorbable Polymer Implant Development

Atorvastatin (ATV) calcium salt and free acid were selected for product development.

The following formulations were prepared with atorvastatin calcium salt and were manufactured using a hot melt extrusion process (Details in Example 2).

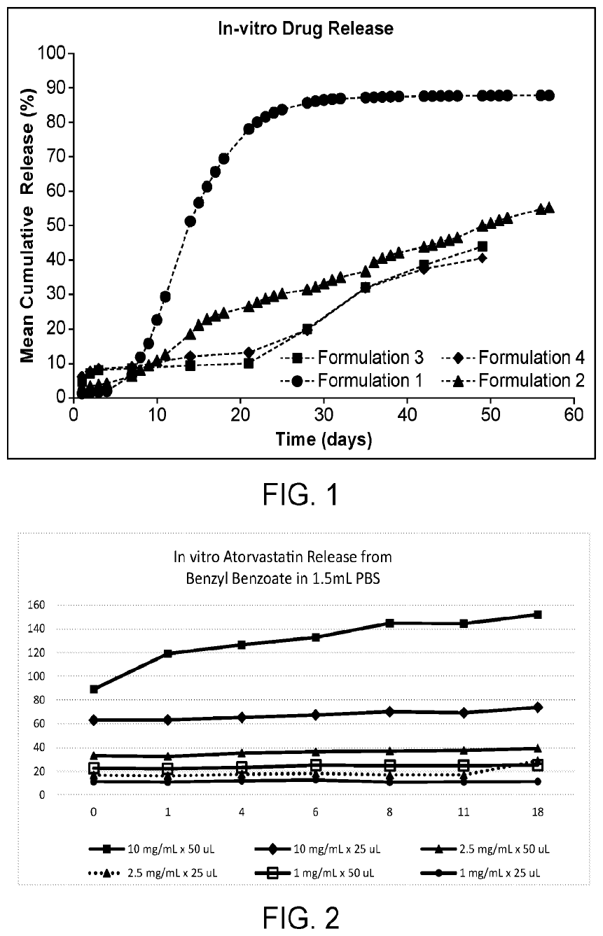

Implant diameter was maintained at 300 μm to allow for administration via a 25G needle. Components are shown in the Table below, and the in vitro release profiles for #3 and #4 are shown in FIG. 1 (DL polymers are PLA polymers composed of D, L-lactide with a racemic mixture of D and L isomers; DLG are PLGA polymers composed of D, L-lactide and glycolide monomers; Ashland).

| | | Molar Ratio | End |
|---|---|---|---|
| Formulation Description | Polymer | (LA:GA) | Group |
| 40% ATV/PLGA (Formulation 1) | DLG | 75:25 | Acid |
| | 7502 A | | |
| 40% ATV/PLGA (Formulation 2) | DLG | 75:25 | Ester |
| | 7502 E | | |
| 40% ATV/PDLLA | DL 03 E | 100:0 | Ester |

A second set of formulations with atorvastatin free acid was manufactured using a hot melt ram extrusion process. The implant diameter was maintained at a value of 250 μm to 300 μm for administration via a 25G needle. Components are shown in the Table below, and the in vitro release profiles are shown in FIG. 1 (RG 653 H refers to Poly(D,L-lactide-co-glycolide) 65:35 ratio of lactide to glycolide; Evonik).

| | | Molar Ratio | End |
|---|---|---|---|
| Formulation Description | Polymer | (LA:GA) | Group |
| 25% ATV/PLGA (Formulation 3) | RG653H | 65:35 | Acid |
| 40% ATV/PLGA (Formulation 4) | RG653H | 65:35 | Acid |

### Example 2: Manufacture of 40% Atorvastatin (ATV) Implants

0.3 mm×6 mm implants were manufactured at a target drug load of 40% ATV using the three selected Viatel polymers: DLG 7502 A, DLG 7502 E, and DL 03 E. Two extrusion cycles were utilized to promote homogeneity of the API in the polymer.

### Materials:

Atorvastatin Calcium Trihydrate,
Milled Viatel DLG 7502 A,
Milled Viatel DLG 7502 E,
Milled Viatel DL 03 E,

### First Extrusion—Compounding Extrusion:

The milled Viatel polymers are removed from the freezer and allowed to reach ambient temperature before opening. Polymer and API are added to a Max 40 speedmixer cup at a target API loading of 40% w/w. The blend is speedmixed for 5 sec. at 2000 rpm, manually shaken/mixed, then speedmixed again for 5 sec. at 2000 rpm. The blend is manually fed into the miniCTW extruder with assistance of the manual feeding piston, extruded at a set screw speed through a 3 mm die, and drawn down to a target OD of 2.5 mm (determined by the conveyer speed). Extruded strands are pelletized to a target length of 2.5 mm (pelletizer pull speed: 1.85, cut speed: 3.95), sealed in a foil pouch with desiccants, and stored in the refrigerator.

### Powder Blend Compositions:

| | DLG | DLG | DL | | |
|---|---|---|---|---|---|
| | 7502 | 7502 | 03 | ATV | Total |
| | A wt. | E wt. | E wt. | wt. | wt. |
| Formulation | (g) | (g) | (g) | (g) | (g) |
| A.) 40% ATV/DLG 7502 A | 9.00 | — | — | 6.00 | 15.00 |
| B.) 40% ATV/DLG 7502 E | — | 9.00 | — | 6.00 | 15.00 |
| C.) 40% ATV/DL 03 E | — | — | 9.00 | 6.00 | 15.00 |

### Compounding Extrusion Parameters:

| | | Extruder | Extruder | Extruder | | Avg. |
|---|---|---|---|---|---|---|
| | Feeder | Temp | Set Speed | Torque | Conveyer | Strand OD |
| Formulation | Speed | (°C) | (rpm) | (Nm) | Speed | (mm) |
| A.) 40% ATV/DLG 7502 A | N/A | 80 −> 100 | 100 | 0.6-1.0 | 5-6 | N/A¹ |
| B.) 40% ATV/DLG 7502 E | N/A | 100 | 100 | 0.6-1.0 | 5-6 | N/A |
| C.) 40% ATV/DL 03 E | N/A | 100 | 100 | 0.6-1.0 | 5-6 | N/A¹ |

### Milling Compounded Pellets:

Compounded pellets were milled to powder form using the rotary grinder (coffee grinder). Milled powder was collected in a sterilization pouch, sealed in a foil pouch with desiccants, and stored in the refrigerator.

### Second Extrusion—Final Strand Extrusion:

The milled API-loaded polymers are removed from the refrigerator and allowed to reach ambient temperature before opening. Material is added to the force feeder hopper (water cooling set temp: 10 °C) and run for ~5-10 minutes at a speed of 100 to prime the screw. The feeder is then attached to the feed port of the extruder. The extruder screws and the force feeder are started simultaneously and material is extruded through a 0.5 mm die. Extruder speed is adjusted as necessary to produce a stable extruder torque and output rate. Once stable, the extruder is switched to “constant torque” mode using the current measured torque. The extruded material is drawn down to the target OD of 0.30 mm (determined by the conveyor speed) and cut to approximately 6″ lengths. After enough in-spec strands are collected, the conveyer speed is decreased to collect some strands at a target OD of 0.40 mm in case they are needed in the future. Extrusion is continued until enough strands are collected for evaluation. Any remaining unused powder is returned to refrigerated storage. Extruded strands from each formulation are collected in 3 plastic storage tubes (0.3 mm strands, 0.4 mm strands, and strands of various OD collected during process adjustments). The tubes containing the strands are sealed in a foil pouch with desiccants and stored in the refrigerator.

| | | | | | Conveyer | |
|---|---|---|---|---|---|---|
| | | | Extruder | Extruder | Speed | Conveyer |
| | Feeder | Extruder | Set Torque | Speed | (0.3 mm | Speed |
| Formulation | Speed | Temp (°C) | (Nm) | (rpm) | OD) | (0.4 mm OD) |
| A.) 40% ATV/DLG 7502 A | 25 | 100 | 0.30 | 29-31 | 8.5 | 6.5 |
| B.) 40% ATV/DLG 7502 E | 25 | 100 | 0.18 | 25-27 | 8.0 | 6.0 |
| C.) 40% ATV/DL 03 E | 25 | 100 | 0.28 | 26-28 | 8.5 | 6.5 |

### Cutting and Packaging Implants:

The extruded strands are removed from the refrigerator and allowed to reach ambient temperature before opening. For each formulation, 0.30 mm OD strands are used to cut n=20 implants to a target length of 6 mm.

### Implant Dimensions (Using USB Digital Microscope):

| 40% ATV/Viatel DLG 7502 A | | | 40% ATV/Viatel DLG 7502 E | | | 40% ATV/Viatel DL 03 E | | |
|---|---|---|---|---|---|---|---|---|
| Implant | | | Implant | | | Implant | | |
| | | Length | | | Length | | | Length |
| ID # | OD (mm) | (mm) | ID # | OD (mm) | (mm) | ID # | OD (mm) | (mm) |
| Avg. (n = 10): | 0.31 | 6.05 | Avg. (n = 10): | 0.30 | 6.01 | Avg. (n = 10): | 0.30 | 6.03 |
| St. Dev.: | 0.01 | 0.07 | St. Dev.: | 0.01 | 0.05 | St. Dev.: | 0.01 | 0.07 |

Implant Weights (Using n=10 Implant Composite):

| | | | Avg. | |
|---|---|---|---|---|
| | | Composite | Implant | ATV per |
| | | Wt. | Wt. | Implant |
| ID | Description | (mg) | (mg) | (μg) |
| A | 40% ATV/Viatel DLG 7502 A | 5.9 | 0.59 | 236 |
| B | 40% ATV/Viatel DLG 7502 E | 5.2 | 0.52 | 208 |
| C | 40% ATV/Viatel DL 03 E | 5.9 | 0.59 | 236 |

### Example 3: Compression Molded Implants

Implant formulations were prepared using various statins as shown in Table 3A, Table 3B, and Table 3C.

TABLE 3A
Implant formulations of Atorvastatin calcium salt

| | Composition (%) | | | | |
|---|---|---|---|---|---|
| Ingredients | F#1 | F#2 | F#3 | F#4 | F#5 |
| Atorvastatin Calcium Trihydrate | 40 | 10 | 40 | 40 | 60 |
| PLGA 5002 A | | 55 | | | |
| PLGA 7502 E | 60 | | 35 | 45 | |
| PLGA 8503 E | | | 25 | 15 | |
| PLA DL 02 A | | 35 | | | 35 |
| PEG 3350 | | | | | 5 |

TABLE 3B
Implant formulations of Rosuvastatin and Pravastatin

| | Composition (%) | | | | | |
|---|---|---|---|---|---|---|
| Ingredients | F#6 | F#7 | F#8 | F#9 | F#10 | F#11 |
| Rosuvastatin | 30 | 30 | 30 | | | |
| Pravastatin | | | | 30 | 30 | 30 |
| PLGA 7502 A | 70 | | | 70 | | |
| PLGA 8503 E | | 70 | | | 70 | |
| PLA DL 03 E | | | 70 | | | 70 |

TABLE 3C
Implant formulations of simvastatin, lovastatin,
mevastatin, pitavastatin and fluvastatin

Composition (%)

| Ingredients | F#12 | F#13 | F#14 | F#15 | F#16 |
|---|---|---|---|---|---|
| Simvastatin | 30 | | | | |
| Lovastatin | | 30 | | | |
| Mevastatin | | | 30 | | |
| Pitavastatin | | | | 30 | |
| Fluvastatin | | | | | 30 |
| PLGA 5002 E | 70 | 70 | 70 | | |
| PLGA 7502 A | | | | 70 | 70 |

Manufacturing process: The first step for manufacturing statin implants involved milling the polymers using a Cryomill (Cole Parmer Freezer Mill). For example, about 1 g of polymer was pre-cooled in a cryovial using liquid nitrogen for 2 min followed by 5 cycles of milling. Each cycle had 30 seconds of milling time and 30 seconds of wait time before the next cycle.

The second step of the manufacturing process involved manufacturing compression molded discs that were pulverized into powder form and further compression molded into an implant. For example, about 60 mg of API (e.g. rosuvastatin, fluvastatin, pravastatin, pitavastatin, mevastatin, simvastatin or lovastatin) and ~140 mg of milled polymer (e.g PLGA 5002A, PLGA 7502E, PLGA 7502A, PLGA 8503E or PLA DL02E) were weighed and manually blended using a metal spatula. About 15 to 20 mg of the material was added to a vacuum compression molding equipment (MeltPrep VCM Pharma Pro) 1 mm disc assembly and heated to a set temperature of ~95 °C to form a compressed disc. This disc was cooled and triturated using a mortar and pestle to give fine powder form. The powdered material was added to a 0.5 mm or 0.8 mm VCM disc tool and compressed into an implant using 95 °C set temperature. The implant was manually cut to ~3 mm length using a blade.

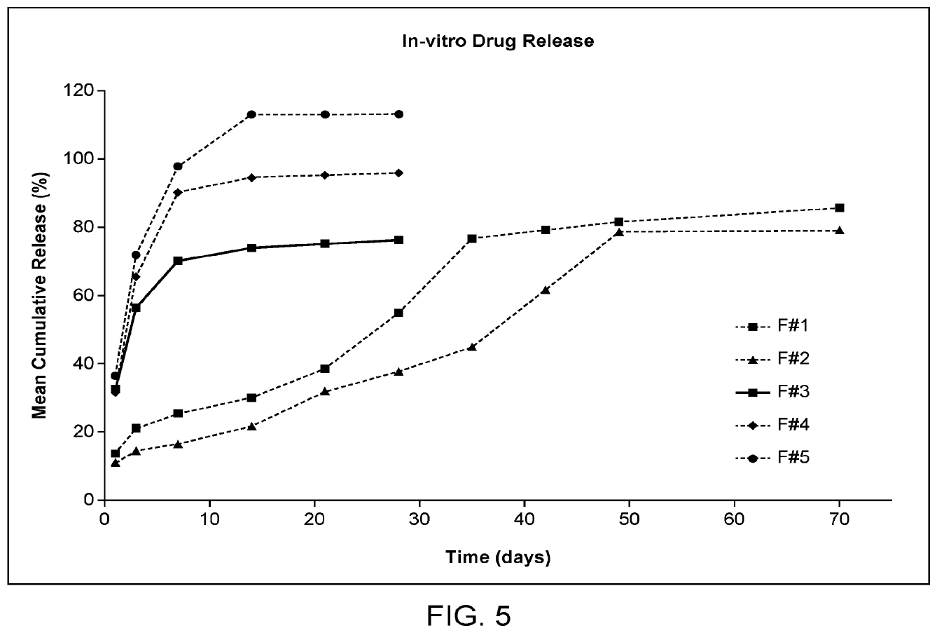

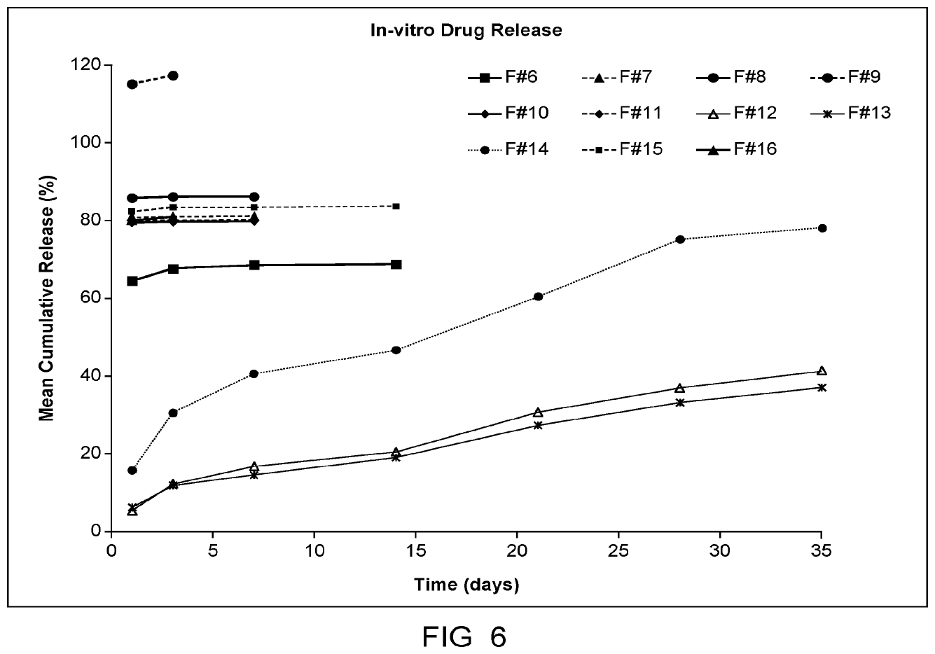

In vitro release (IVR) testing was performed to assess release duration of statin from the implant formulations. FIG. 5 and FIG. 6 show in vitro release of the statin formulations listed in Table 3A, Table 3B, and Table 3C.

### Example 4: Extruded Implants

Atorvastatin (free acid or calcium salt) implant formulations as shown in Table 4 were made and tested to assess their in vitro release properties.

TABLE 4
Implant formulations of atorvastatin free acid or calcium salt.

| | Composition (%) | | | | | | | | | |
|---|---|---|---|---|---|---|---|---|---|---|
| Ingredients | F#17 | F#18 | F#19 | F#20 | F#21 | F#22 | F#23 | F#24 | F#25 | F#26 |
| Atorvastatin | 40 | 40 | 40 | | | | 40 | 40 | | 40 |
| calcium | | | | | | | | | | |
| trihydrate | | | | | | | | | | |
| Atorvastatin free | | | | 25 | 25 | 40 | | | 40 | |
| acid | | | | | | | | | | |
| PLGA 5002 E | | | | | | | | | | 60 |
| PLGA RG653 H | | | | 75 | | 60 | | | | |
| PLGA 7502 A | 60 | | | | | | | 60 | | |
| PLGA 7502 E | | 60 | | | | | 60 | | 60 | |
| PLGA RG752 S | | | | | 75 | | | | | |
| PLA DL 03 E | | | 60 | | | | | | | |

Manufacturing process: The manufacturing process for atorvastatin implants involved polymer-milling, blending, first extrusion, pelletizing, second extrusion, implant cutting, and sterilization (7502 E=Viatel from Ashland; RG 752 S=Resomer from Evonik).

For example, as a first step in implant manufacturing process, the polymers were milled using a Cryomill (Retsch CryoMill). About 15 g of polymer was pre-cooled in a cryovial using liquid nitrogen for 2 mins followed by 5 cycles of milling. Each cycle had 30 secs of milling time and 30 secs of wait time before the next cycle.

The second step of the manufacturing process involved blending the polymer with API. About 10 g of atorvastatin and 14 g of PLGA polymer were added to a jar and blended using Turbula mixer for 10 min at 20 rpm. The powder blend was extruded at a barrel temperature of 100 °C and screw speed of 30 rpm using ~2 mm die. The extruded filaments were pelletized using a 25 mm milling ball and further extruded at a barrel temperature of 100 °C and screw speed of 30 rpm using 0.3 mm die. The filaments were screened for diameter using a laser micrometer and manually cut to 6 mm implant length using a cutting fixture.

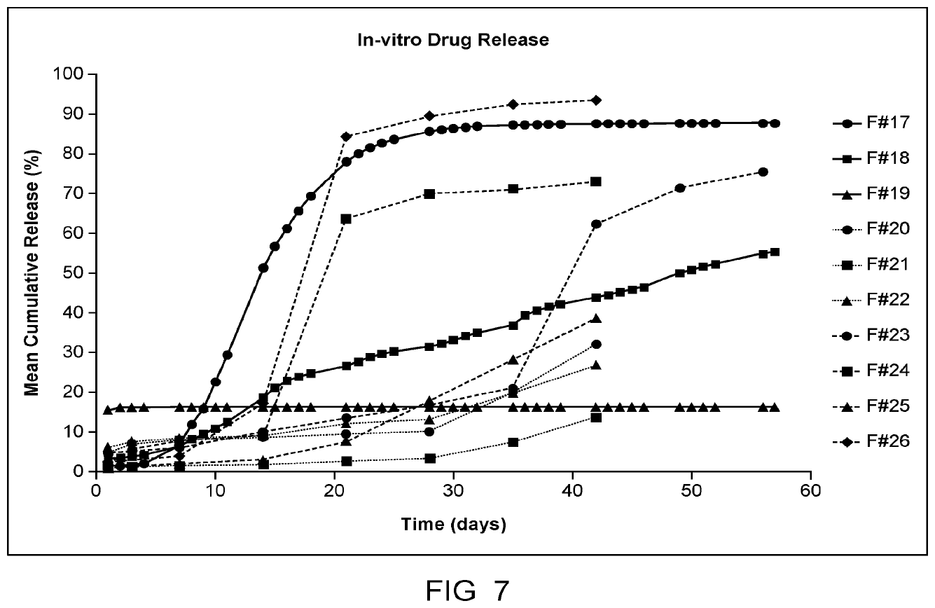

In vitro release (IVR) testing was performed to assess release duration of statin from the implant formulations. FIG. 7 shows the in vitro release of atorvastatin from implant formulations listed in Table 4.

### Example 5: Atorvastatin Benzyl Benzoate Composition

Atorvastatin free acid was found to be soluble in benzyl benzoate. Solution formulations of various concentrations were prepared by placing 10 g of benzyl benzoate in 20 mL scintillation vials and adding a specified amount of atorvastatin free acid to the same vial. A vortex mixer was used to mix and dissolve the atorvastatin in benzyl benzoate. Solutions concentrations prepared ranged from 1 mg/mL to 10 mg/mL atorvastatin in benzyl benzoate.

In vitro drug release rate studies were performed by placing 25 μL or 50 μL of the atorvastatin solution in a vial containing buffer and drawing a sample from the vial at various time points and determining the concentration of atorvastatin in the buffer 18-day data indicate that sustained release of atorvastatin is feasible and tunable based on concentration of drug in benzyl benzoate and droplet volume of the formulation. Results are shown in FIG. 2.

### Example 6: Drusen In Vitro Assay

Retinal Pigmented Epithelium Cell Culture: Human embryonic stem-cell derived retinal pigmented epithelium (hESC-RPE) were cultured on Transwell inserts for at least 4 weeks in serum-free XVIVO culture medium. After 4 weeks, polygonal, pigmented monolayers of hESC-RPE were visualized by phase contrast and bright-field microscopy, indicating appropriately matured RPE cells. Drusen assay experiments were performed after the initial 4-week maturation period.

Drusen Assay: Matured hESC-RPE monolayers were incubated with 5% complement competent human serum (HS) for 48 hours to elicit Drusen formation. After the 48 hour HS incubation period, hESC-RPE monolayers (whole inserts) were fixed with 4% paraformaldehyde for 10 minutes at room temperature. Following fixation, inserts were individually removed from plates and cut out of their holders using a scalpel yielding individual membranes with hESC-RPE monolayers. Membranes were transferred to 24-well plates for storage in phosphate buffered saline with calcium and magnesium (PBS+/+).

Drusen Assay with Atorvastatin Treatment: Matured hESC-RPE inserts were pre-incubated with varying concentrations of atorvastatin (ranging from 100 μM to 10 μM) in both the top and bottom insert compartments for 24 hours in an incubator set at 37 °C with 5% CO₂. On the following day, medium was exchanged with fresh culture medium containing atorvastatin and 5% HS and incubated for an additional 48 hours in an incubator set at 37 °C with 5% CO₂. After 48 hour incubation period, inserts were fixed and processed for antibody staining.

### Antibody Staining and Imaging

Fixed membranes were incubated with block solution containing 1% bovine serum albumin (BSA) and 0.1% Triton X in PBS+/+ for 1 hr at room temperature. After the block incubation period, membranes were incubated overnight at 4 °C with primary antibody solution containing 1% BSA and goat anti-APOE4 antibody (1:250) and mouse anti-C5b9 antibody (1:250). On the following day, membranes were washed thrice for 10 min with PBS+/+ and incubated with secondary antibody solution containing 1% BSA and AlexaFluor donkey anti-goat 546 (1:200) and AlexaFluor donkey anti-mouse 488 (1:200) for 1 hr at room temperature in the dark. Following secondary antibody incubation period, membranes were washed thrice for 10 min with PBS+/+ and counterstained with Hoechst solution (1:1000) in PBS+/+ for 10 min at room temperature. Membranes were then transferred to microscope slides and mounted using ProLong Gold anti-fade solution and coverslips. Slides were imaged using a Leica SP8 Scanning Resonant Confocal Microscope using identical settings and laser power. At least 3 fields of view were collected per sample.

Image quantification and figure preparation: Fiji (Image J) software was used for processing all the images. All images were processed with the parameters described below: Contrast adjustment—the value of contrast for the APOE4 channel was set to 90-230 to improve the accuracy of object observation. Thresholding—the cutoff value of the threshold for the APOE4 channel was set as 100 to 255 to identify the pixels as real expressions. Size-Particles with an area larger than 1.5 A.U. were counted as true APOE4 expressions to minimize the likelihood of false positives in the image. Measurements, including deposit counts and the total area, were then exported as a .csv file for analysis. All images were taken in triplicate per treatment. Results are presented as mean±SEM and were graphed using Graph Pad PRISM v6. Statistical significance was established using one-way ANOVA tests followed by Tukey's multiple comparisons test.

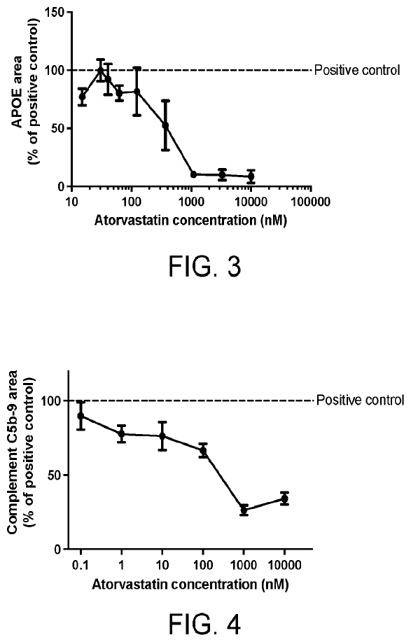

Drusen (APOE4) results are shown in FIG. 3.

Atorvastatin (ATV) reduced the total counts of APOE deposition dose-dependently. The total counts of APOE deposits decreased starting at 123 nM of ATV treatment with nearly complete elimination at higher ATV concentrations.

Atorvastatin reduces APOE4 and C5b-9 expression in hESC-RPE treated with human serum. Representative immunofluorescence images of hESC-RPE treated with 5% human serum and varying concentrations of Atorvastatin. Increasing concentrations of atorvastatin results in decreased expression of APOE4 (FIG. 3) and C5b-9 positive puncta (FIG. 4) per field of view.

Atorvastatin (ATV) reduced APOE deposition dose-dependently. ES-derived RPE cells were treated with 5% Human serum to induce APOE deposition, which area decreased upon atorvastatin treatment. APOE deposition area decreased starting at 370 nM of ATV treatment with nearly complete elimination at higher ATV concentrations.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

What is claimed is:

1. An ocular implant comprising a statin and a polymer matrix, wherein the statin is dispersed in the polymer matrix and the polymer matrix controls release of the statin.

2. The ocular implant of claim 1, wherein the implant has a length of from about 1 mm to about 10 mm.

3. The ocular implant of claim 1 or 2, wherein the implant has a diameter of from about 100 μm to about 0.5 mm.

4. The ocular implant of any one of claims 1-3, wherein the implant is a cylindrical pellet.

5. The ocular implant of any one of claims 1-4, wherein the polymer matrix is swellable in vivo.

6. The ocular implant of any one of claims 1-5, wherein the implant is biodegradable.

7. The ocular implant of any one of claims 1-5, wherein the implant is non-biodegradable.

8. The ocular implant of any one of claims 1-7, wherein the implant comprises from about 30 wt % to about 90 wt % polymer.

9. The ocular implant of any one of claims 1-8, wherein the implant comprises from about 10 wt % to about 70 wt % statin.

10. The ocular implant of any one of claims 1-9, wherein the polymer matrix comprises poly(DL-lactide), poly(DL-lactide-co-glycolide), or a mixture thereof.

11. The ocular implant of any one of claims 1-10, wherein the polymer matrix is a 50/50, 65/35, 75/25, 85/15, 90/10, or 95/5 poly(DL-lactide-co-glycolide), poly(DL-lactide), or a mixture thereof.

12. The ocular implant of any one of claims 1-11, wherein the polymer matrix is an acid or ester end-capped poly(DL-lactide) polymer or acid or ester end-capped poly(DL-lactide-co-glycolide), or a mixture thereof.

13. The ocular implant of any one of claims 1-12, wherein the polymer matrix has an inherent viscosity of about 0.05 to about 1.7 dL/g.

14. An ocular implant comprising a statin selected from the group consisting of atorvastatin, rosuvastatin, pravastatin, pitavastatin, simvastatin, lovastatin, mevastatin, and fluvastatin, or a salt thereof; and a polymer matrix comprising acid or ester end-capped poly(DL-lactide) polymers or acid or ester end-capped poly(DL-lactide-co-glycolide) 50/50, 65/15, 75/25, 85/15 polymers; wherein the statin is dispersed in the polymer matrix and the polymer matrix controls release of the statin.

15. The ocular implant of claim 14, wherein the polymer matrix comprises PLGA 5002 A, PLGA 5002 E, PLGA 7502 A, PLGA 7502 E, PLGA 8503 E, PLGA RG653 H, PLGA RG752 S, PLA DL 02 A, PLA DL 03 E, PLGA 7502 A, or PLA DL 03 E, or PEG 3350, or a mixture thereof.

16. The ocular implant of claim 14 or 15, wherein the implant comprises from about 10 wt % to about 40 wt % statin.

17. A flowable composition comprising a statin and a solvent or excipient, wherein the statin is dispersed in a solvent or excipient and the solvent or excipient controls release of the statin.

18. The flowable composition of claim 17, wherein the solvent or excipient is benzyl benzoate.

19. An extended release unit dosage form for ocular administration of a statin comprising the ocular implant of any one of claims 1-16 or the composition of claim 17 or 18.

20. The extended release unit dosage form of claim 19, wherein the statin is selected from the group comprising fluvastatin, lovastatin, pravastatin, atorvastatin, simvastatin, cerivastatin, rivastatin, mevastatin, velostatin, compactin, dalvastatin, fluindostatin, rosuvastatin, and pitivastatin, or a pharmaceutically acceptable salt thereof.

21. The extended release unit dosage form of claim 20, wherein the statin is atorvastatin or a salt thereof.

22. The extended release unit dosage form of claim 21, wherein the statin is atorvastatin free acid.

23. The extended release unit dosage form of claim 21, wherein the statin is atorvastatin calcium salt.

24. The extended release unit dosage form of claim 23, wherein the atorvastatin calcium salt is present in particles, dispersed in the polymer matrix or the benzyl benzoate.

25. The extended release unit dosage form of any one of claims 19-24, wherein the implant or composition is configured to substantially maintain an intravitreal concentration of statin of from about 10 nM to about 5 μM over the course of a treatment period.

26. The extended release unit dosage form of claim 25, wherein the treatment period is 1-30 days, 4-52 weeks, 1-12 months, or 1-5 years.

27. A method of treating or preventing age-related macular degeneration (AMD), comprising administering a statin to a patient in need thereof, wherein the statin is administered to the eye in an extended release unit dosage form, such that the statin concentration within the eye is substantially maintained at a concentration of greater than about 10 nM for a treatment period of least 1 month.

28. A method of treating or preventing a retinal disease or disorder, comprising administering the extended release unit dosage form of any one of claims 19-26, to a patient in need thereof.

29. The method of claim 28, wherein the retinal disease or disorder is age-related macular degeneration, macular drusen (small, intermediate, large), peripheral drusen, extramacular drusen, drusenoid pigment epithelial detachment (PED), drusenoid deposits, basal laminar deposits, basal linear deposits, doyne honeycomb retinal dystrophy, Malattia Leventinese, familial dominant drusen (or autosomal dominant drusen), cuticular drusen, serous detachment of RPE, drupelets, RPE atrophy, geographic atrophy, ellipsoid zone (EZ) attenuation, EZ loss, incomplete retinal pigment epithelial and outer retinal atrophy (iRORA), complete retinal pigment epithelial and outer retinal atrophy (cRORA), nascent geographic atrophy, retinal flecks, fundus flavimaculatus, Best disease, adult-onset vitelliform macular dystrophy, Best vitelliform macular dystrophy, autosomal recessive bestrophinopathy, vitelliform material, pattern dystrophy, autosomal dominant vitreoretinochoroidopathy, BEST1 gene mutation disorders, retinal emboli (in retinal artery occlusion), retinal exudates, retinal exudates secondary to retinal microaneurysm, familial exudative vitreoretinopathy (FEVR), synchysis scintillans (cholesterolosis bulbi), neuronal ceroid lipofuscinosis, Batten's Disease, retinitis pigmentosa, Bietti's crystalline dystrophy, juvenile macular degeneration (e.g., Stargardt disease), macular telangiectasia, maculopathy (e.g., age-related maculopathy (ARM) and diabetic maculopathy (DMP) (including partial ischemic DMP)), macular edema (e.g., diabetic macular edema (DME) (including clinically significant DME, focal DME and diffuse DME), Irvine-Gass Syndrome (postoperative macular edema), and macular edema following RVO (including central RVO and branch RVO)), retinopathy (e.g., diabetic retinopathy (including in patients with DME), Purtscher's retinopathy and radiation retinopathy), retinal artery occlusion (RAO) (e.g., central and branch RAO), retinal vein occlusion (RVO) (e.g., central RVO (including central RVO with cystoid macular edema (CME)) and branch RVO (including branch RVO with CME)), glaucoma (including low-tension, normal-tension and high-tension glaucoma), ocular hypertension, retinitis (e.g., Coats' disease (exudative retinitis) or retinitis pigmentosa), chorioretinitis, choroiditis (e.g., serpiginous choroiditis), uveitis (including anterior uveitis, intermediate uveitis, posterior uveitis with or without CME, and pan-uveitis), retinal detachment (e.g., in von Hippel-Lindau disease), retinal pigment epithelium (RPE) detachment, bestrophinopathy, Doyne honeycomb/dominant drusen, and diseases associated with increased intra- or extracellular lipid storage or accumulation in addition to AMD.

30. The method of claim 28, wherein the administering is by intravitreal injection.

31. A method of treating or preventing age-related macular degeneration (AMD) comprising administering a statin to a patient in need thereof, wherein the administering is by intravitreal injection.

32. A method of reducing drusen size and/or number comprising administering a statin to a patient in need thereof, wherein the administering is by intravitreal injection.

33. A method of preventing, reducing, or reversing complement activation in the eye comprising administering a statin to a patient in need thereof, wherein the administering is by intravitreal injection.

34. The method of any one of claims 27-33, wherein the statin is administered in an interval of once per month, once per three months, once per six months, or once per year.

35. A method of treating or preventing age-related macular degeneration (AMD), comprising administering an ultralow daily dose of statin to a patient in need thereof over the course of a treatment period.

36. The method of claim 35, wherein the statin is administered to an eye of the patient as an extended release unit dosage form.

37. The method of claim 35 or 36, wherein the ultralow daily dose of statin per eye is about 20 μg or less.

38. The method of claim 35 or 36, wherein the ultralow daily dose of statin per eye is from about 0.01 μg to about 20 μg.

39. The method of any one of claims 35-38, wherein the treatment period is 1-30 days, 4-52 weeks, 1-12 months, or 1-5 years.
